(19) United States
(12) Patent Application Publication
(10) Pub. No.: US 2025/0361349 A1
(43) Pub. Date: Nov. 27, 2025

(54) MONOMER MIXTURE FOR PRODUCING A DENTAL MATERIAL

(71) Applicant: Mühlbauer Technology GmbH, Hamburg (DE)

(72) Inventors: Stephan Neffgen, Hamburg (DE); Swen Neander, Hamburg (DE)

(21) Appl. No.: 18/881,158

(22) PCT Filed: Jun. 30, 2023
(86) PCT No.: PCT/EP2023/067958

§ 371 Date: Jan. 3, 2025

(30) Foreign Application Priority Data
Jul. 4, 2022 (DE) 10 2022 116 577.9

(51) Int. Cl. C08F 283/00; A61K 6/887; C08F 2/50

(52) U.S. Cl. CPC C08F 283/008; A61K 6/887; C08F 2/50

(57) ABSTRACT

The invention relates to a monomer mixture for producing a dental material, comprising: a. at least one base monomer M1 of the following empirical formula 1: KₙUₘ(O—S-PG), (formula 1), b. at least one base monomer of the following formula 4: PG′-S′-A′-S′-PG′ (formula 4), and a monomer mixture for producing a dental material, comprising at least one or more base monomers M1 of the empirical formula 1, the use of the monomer mixtures, polymerizable dental materials containing such monomer mixtures, polymerizable dental materials for use in a therapeutic method and cured dental materials.

The invention relates to a monomer mixture for producing a dental material, to a use of the monomer mixture, to a polymerizable dental material comprising such a monomer mixture, to a polymerizable dental material comprising such a monomer mixture for use in a treatment process, and to a cured dental material.

Radically polymerizable dental materials mainly comprise (meth)acrylate monomers. Restorative and prosthetic dental materials such as dental fillings or dentures generally employ dimethacrylate systems on account of their properties such as rapid free-radical polymerization, good mechanical properties, and esthetic appearance. Customary base monomers are high-molecular-weight structures containing linear aliphatic or aromatic groups and having terminal methacrylate functionalities, for example 2,2-bis[4-(2-hydroxy-3-methacryloyloxypropoxy)phenyl]propane (bis-GMA) and 7,7,9-trimethyl-4,13-dioxo-3,14-dioxa-5,12-diazahexadecane-1,16-diyl bis(2-methylacrylate) (UDMA).

There have for some time been efforts to cease using bis-GMA and to replace it, to some degree at least, with other compounds. The spotlight here has been on urethane monomers and oligomers in particular. In the field of dental materials the substance with most widespread commercial use as an at least partial substitute for bis-GMA is UDMA.

Base monomers such as bis-GMA and UDMA, although present in broad ranges of commercial radically polymerizable dental materials, have some drawbacks. They are generally highly viscous to solid substances. Mixtures with monomers having significantly low viscosity, such as triethylene glycol dimethacrylate (TEGDMA), are accordingly used. TEGDMA is a very versatile, low-molecular-weight monomer with low viscosity (of 0.01 Pa·s) and has high mobility during polymerization, which facilitates conversion in the polymerization.

However, these monomer mixtures and the dental materials obtained therefrom have some problematic properties that can adversely affect clinical treatment outcomes. For example, monomer mixtures of these dimethacrylate monomers display relatively low polymerization conversions, substantial polymerization shrinkage, poor toughness, and undesirable water absorption. The known systems are often able to achieve only a comparatively low conversion of the double bonds, which not only contributes to inadequate mechanical properties and poor wear resistance, but is also disadvantageous in respect of the toxicology and biocompatibility of the polymerized dental materials. In addition, the volume shrinkage of the currently used dimethacrylate monomers and the shrinkage stresses of a tooth filling can result in failure of the bond between tooth and filling, leading to microleaks and consequently to secondary caries, which in turn can significantly reduce the longevity of the restoration. Attempts to boost the double bond conversion so as to reduce the unreacted monomer content unfortunately lead to an increase in polymerization shrinkage and shrinkage stress.

Low-molecular-weight monomers having oligo(ethylenoxy) groups, such as TEGDMA, which have a degree of solubility in water and thus bioavailability, are now being evaluated critically on account of their toxicological properties and susceptibility to biodegradation processes. Monomers containing the bis-2,2[p-oxyphenyl]propane structural element, i.e. monomers based on bisphenol A, are likewise being evaluated critically, since dental materials comprising monomer mixtures containing these structural elements have been found to release detectable amounts of bisphenol A, to which toxicologically critical properties are attributed.

There are various approaches to increasing conversion or reducing volume shrinkage. In dental composites for dental fillings, which comprise filler in a matrix of organic resin, attempts are being made to reduce volume shrinkage by increasing the filler content. However, if the filler content is too high, it is difficult to mix the fillers with the organic resin. In addition, there is a limit to the amount of filler that is possible in a dental composite. The option of reducing polymerization shrinkage by increasing the filler content is thus fundamentally limited.

To increase conversion and reduce polymerization shrinkage, there is ongoing development of novel monomers, for example high-molecular-weight urethane methacrylate monomers. Increasing the molecular weight is usually associated with poorer mechanical properties in the cured dental materials for a given monomer functionality. Moreover, the increased viscosity of such monomers means they must be used alongside higher amounts of low-viscosity monomers in order to permit use in dental composites, which has an adverse effect on shrinkage.

EP 2436365 B1 describes low-shrinkage dental composites comprising monomer mixtures in which the monomers (b1) and (b2) are present in a ratio of 1:20 to 5:1. The example compositions contain in each case 4.8-76.6% by weight of bis((meth)acryloyloxymethyl)tricyclo[5.2.1.0^{2,6}]decane (b1), 90.9-19.1% by weight of UDMA (b2), and 4.3% by weight of TEGDMA (b2). These composites display a polymerization shrinkage of about 1.50% irrespective of the ratio of (b1) to (b2). When, as in comparative example 11, the filler content is reduced and the proportion of TEGDMA is increased, the polymerization shrinkage increases.

Vaidyanathan et al. “Visible light cure characteristics of a cycloaliphatic polyester dimethacrylate alternative oligomer to bisGMA”, Acta Biomater Odontol Scand. 2015; 1:59-65, disclose the use of PEM-665 as a BPA-free alternative to bis-GMA in combination with 30% and 50% by weight of TEGDMA. Investigation of the polymerization conversion of these mixtures found that the combinations of PEM with TEGDMA showed a higher percentage polymerization conversion than the combinations of bis-GMA with TEGDMA.

U.S. Pat. No. 4,554,336 describes orthodontic adhesives based on trifunctional polyether urethane (alk)acrylates having a nonlinear structure. However, the urethane acrylates with a nonlinear structure from U.S. Pat. No. 4,554,336 result inter alia in dental composites having a reduced elastic modulus.

There is therefore a need for monomers or monomer mixtures that can permit a reduced toxicity potential and reduced volume shrinkage alongside good mechanical properties in the dental material, in particular dental restoration and filling material, to be produced therefrom and that are readily available.

The object of the present invention is thus to provide a monomer mixture that overcomes the abovementioned disadvantages of the prior art and that in particular makes it possible to produce dental materials, in particular dental composites, having improved volume shrinkage, improved flexural strength, and a good elastic modulus.

The invention achieves this object through a monomer mixture for producing a dental material, comprising:
a. at least one base monomer M1 of the following empirical formula 1:

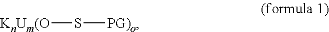

where
   PG=is a polymerizable group selected from —OOC—CH═CH₂ and —OOC—C(CH₃)═CH₂;
   S=is a spacer group selected from unbranched and branched alkylene having 1-10 carbon atoms, which may additionally contain oxygen and/or —OOC— in the carbon chain, and is preferably ethylene;
   O=is oxygen;
   U=is a group represented by the following formula 2:

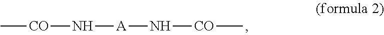

where
   A=is a group selected from a divalent aromatic or aliphatic C6-C20 hydrocarbon group, preferably a divalent C6-C13 aliphatic hydrocarbon group, more preferably a divalent saturated, cyclic C6-C13 hydrocarbon group;
   K=is a group represented by the following formula 3:

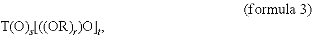

where
   T=is a trivalent hydrocarbon group having 3-7 carbon atoms,
   O=is oxygen,
   R=is in each case independently selected from an ethylene group, a 1,2-propylene group, a 1,3-propylene group and a mixture thereof, preferably a 1,2-propylene group,
   r=is in each case independently 1-12, preferably 1-9, more preferably 1-6,
   s=is 0 or 1, preferably 0,
   t=is 2 or 3, preferably 3,
   where the condition must be met that s+t=3;
   n=1-9, preferably 1-7, more preferably 1-5;
   where the condition must be met that m=2n+1 and o=n+2;
b. at least one base monomer M2 of the following formula 4:

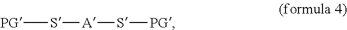

where
   PG′=is a polymerizable group selected from —OOC—CH═CH₂ and —OOC—C(CH₃)═CH₂;
   S′=is a spacer group selected from unbranched and branched alkylene having 1-10 carbon atoms, which may additionally contain oxygen and/or —OOC— in the carbon chain, and is preferably methylene;
   or S′ is absent;
   A′=is an aliphatic polycyclic group, preferably an aliphatic tricyclic hydrocarbon group, in which one or more hydrogen atoms may each be independently replaced by C1-C4 alkyl radicals, C1-C4 alkoxy radicals, fluorine atoms, chlorine atoms or trifluoromethyl groups, more preferably is tricyclodecanylene, even more preferably tricyclo[5.2.1.0^{2,6}]decanylene.

Preferred embodiments can be found in the subclaims.

First of all, some terms used in the context of the invention will be explained.

In accordance with the invention, polymerizable dental materials are understood as meaning materials for (bio) medical use, in particular on dental hard substance, such as enamel and dentine, or on bone tissue, such as on the jawbone.

The polymerizable dental material is usually a resin-based material comprising a curable mixture of various constituents. In the context of the present invention, a resin essentially consists of the monomer mixture and further constituents soluble in the monomers, for example initiators, stabilizers, etc.

In the context of the present invention, a monomer mixture is a mixture comprising base monomers M1 and M2 and optionally base monomers M3 and/or other monomers (OM) of the polymerizable dental material. Further constituents of the polymerizable dental material such as initiators, fillers, customary dental additives, etc. are not constituents of the monomer mixture.

In the context of the present invention, base monomer M1 is a monomer when n=1 and oligomers when n=2 to 9. In the present case, monomers and oligomers where n=1 to 9 are also referred to as base monomers M1.

In the context of the invention, T from formula 3 is a trivalent hydrocarbon group having C3-C7 carbon atoms. In the context of the invention, trivalent means that three bonds start from group T, these bonds preferably starting from three different carbon atoms. Preferably, T is a carbon radical derived from glycerol, 2-ethyl-2-(hydroxymethyl) propane-1,3-diol, hexanetriol (1,2,6-isomer, 1,3,5-isomer, 1,2,3-isomer, 2,3,5-isomer, and mixtures thereof), butanetriol, 2-(hydroxymethyl) propane-1,3-diol, 2-methylpropane-1,2,3-triol, pentanetriol, 2-(hydroxymethyl) butane-1,4-diol, 2-(hydroxymethyl) butane-1,3-diol, 3-methylpentane-1,3,5-triol, 2-(hydroxymethyl) hexane-1,6-diol, and 3-(hydroxymethyl) hexane-1,6-diol. In addition, the C3-C7 carbon radical may also be derived from any other trifunctional alcohols from the prior art. More preferably, T is a trivalent hydrocarbon group having 3 carbon atoms. In a preferred embodiment, T is represented by the following formula 5:

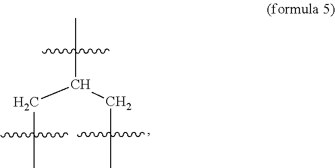 [SMILES: *CC(*)C*]

where the points of attachment to the oxygen atoms in formula 3 are each represented by the indicated bond (i.e. the broken lines).

In the context of the invention, A from formula 2 is a group selected from a divalent aromatic or aliphatic C6-C20 hydrocarbon group. In the context of the invention, divalent means that two bonds start from group A, these bonds preferably starting from two different carbon atoms. Preferably, A is a divalent aliphatic C6-C13 hydrocarbon group, more preferably a divalent saturated cyclic C6-C13 hydrocarbon group. Preferably, A is a divalent cyclic hydrocarbon group having 10 carbon atoms. In a preferred embodiment, A is represented by the following formula 6:

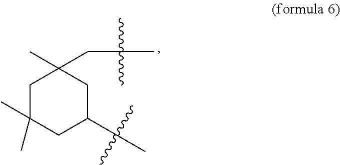 [SMILES: *CC1(C)CC(*)CC(C)(C)C1]

where the two indicated bonds (i.e. the broken lines) each represent the points of attachment to the nitrogen atoms in formula 2.

The monomer mixture preferably comprises a plurality of base monomers M1, more preferably at least two base monomers M1, even more preferably more than two base monomers M1, further preferably more than three base monomers M1, even further preferably more than four base monomers M1, even further preferably more than five base monomers M1, and so on. Where the monomer mixture does comprise a plurality of base monomers M1, it is preferably a mixture that, in addition to base monomers, also includes base oligomers. In accordance with the invention, such a mixture is also referred to as a mixture of base monomers M1.

It is preferable that, in addition to the base monomer M1 where n=1 (i.e. a monomer), at least one base monomer M1 where n is greater than 1 (i.e. an oligomer) is also present. The mass fraction of the base monomer(s) M1 where n is greater than 1, which can be determined through fractionation by gel-permeation chromatography using a refractive index detector (measurement with visible light), is preferably 5-70% by weight, more preferably 10-60% by weight, even more preferably 15-50% by weight, based on the total mass of all base monomers M1 of a monomer/oligomer series of n=1-9.

The distribution of the base monomers M1 in respect of n can vary within wide ranges. It may be the case that the base monomers M1 in which n=2-5 or n=2-4 have the highest mass fraction based on the total mass fraction of the base monomers M1. However, it may also be the case that the base monomer(s) M1 where n=1 have the highest mass fraction compared to each individual base monomer M1 present in the monomer mixture where n=2-9.

Some structures of the base monomer M1 for various n are outlined below. In the case where n=1, observance of the conditions m=2n+1 and o=n+2 gives the empirical formula K₁U₃ (O—S-PG)₃ for the base monomer M1. The combination of the trivalent group K with the divalent group U and the monovalent group —O—S-PG gives the structure for the base monomer M1 shown in formula 7 below for n=1:

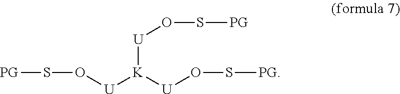

In the case where n=2, this gives the empirical formula K₂U₅ (O—S-PG) 4 for the base monomer M1. In this case, the base monomer M1 can be represented by the structure shown in formula 8:

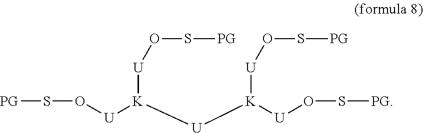

In the case where n=3, this gives the empirical formula K₃U₇(O—S-PG) 5 for the base monomer. In this case, the base monomer M1 can be represented by the structure shown in formula 9:

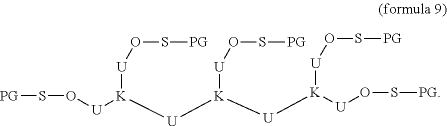

In the case where n=4, this gives the empirical formula K₄U₉ (O—S-PG) 6 for the base monomer. For n=4, the base monomer M1 can already be represented by two different structures, which are shown in formulas 10 and 11 below:

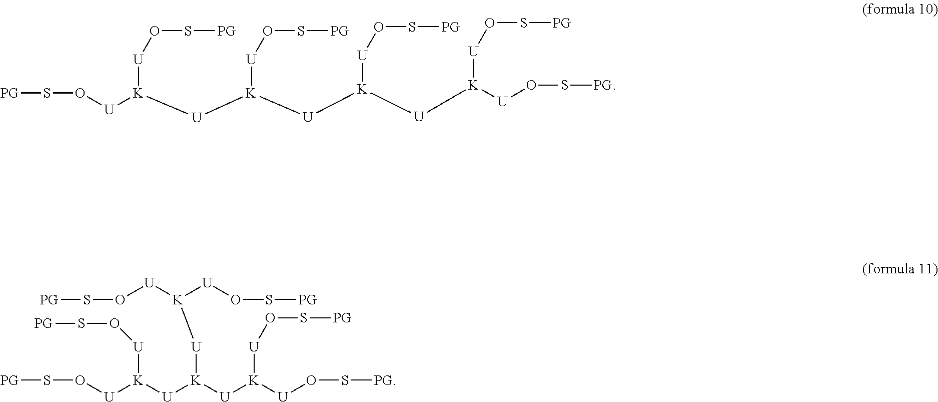

In the case where n=5, this gives the empirical formula K₅U₁₁ (O—S-PG) 7 for the base monomer. For n=5, the base monomer M1 can likewise be represented by two different structures, which are shown in formulas 12 and 13 below:

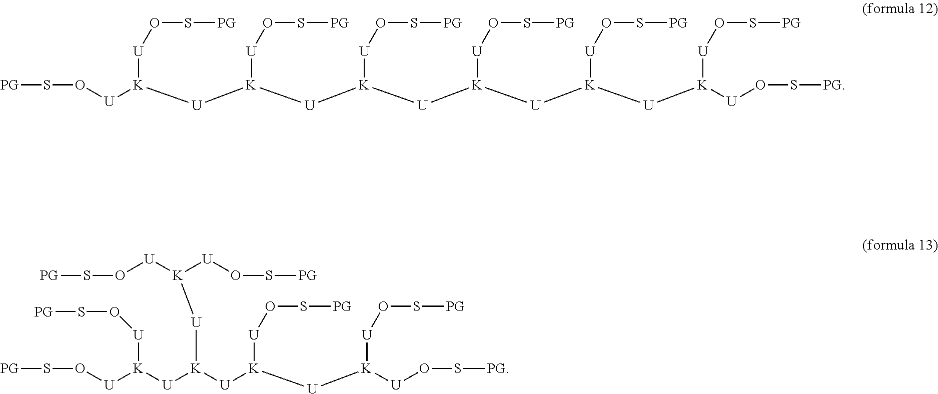

In the cases in which n=6-9, the number of possible structures then increases for each additional n.

Specifically, within group K the three carbon atoms of group T are each attached to corresponding oxygen atoms T(O)ₛ[((OR)ᵣ)O]ₜ or T(O)ₛ[((OR)ᵣ)O]ₜ. Group K can be attached to carbamoyl carbon atoms —CO—NH— of group U via the indicated oxygen atoms T(O)ₛ[((OR)ᵣ)O]ₜ or T(O)ₛ[((OR)ᵣ)O]ₜ. The carbamoyl carbon atoms —NH—CO— of group U can be attached either to an oxygen atom of the —O—S-PG group and to an oxygen atom of group K(T(O)ₛ[((OR)ᵣ)O]ₜ or T(O)ₛ[((OR)ᵣ) O]ₜ), or be attached to two oxygen atoms of different groups K. The oxygen atom of the —O—S-PG group is always attached to a carbamoyl carbon atom NH—CO— of group U.

The base monomers M1 where n>1 are present as base monomers where n=2-9, preferably where n=2-7, more preferably where n=2-5.

In one embodiment, all base monomers M1 of a monomer/oligomeric series where n=1-9, preferably where n=1-7, more preferably where n=1-5, may be present side by side. This means that if at least one compound in each case is present for each n from 1-9, then at least 9 compounds (i.e. a monomer for n=1 and eight oligomers for n=2, 3, 4, etc.) would be present in the monomer mixture (or monomer/oligomer mixture). For n=1-7 there would then be at least 7 compounds (i.e. a monomer and six oligomers) and for n=1-5 there would be at least 5 compounds (i.e. a monomer and four oligomers) present in the monomer mixture.

In the context of the invention, r is in each case independently 1-12, preferably 1-9, even more preferably 1-6. Since it is possible for t to vary in the base monomer M1, i.e. for t to be 2 or 3, the number of radicals r present varies accordingly too, i.e. r can in the context of the invention correspond to either the radicals r1 and r2 or to the radicals r1, r2, and r3. In the context of the invention, the radicals r1-r2 or r1-r3 in a base monomer M1 may each be identical or they may differ from one other.

That is to say, the groups K may be distributed by molecular weight, because the —O—R— groups may be present side-by-side with different stoichiometric indices r1, r2, r3.

The sum of the coefficients r1, r2, r3 is preferably greater than 3. In a preferred execution, r1+r2+r3=4-20.

The base monomer M2 may be selected from bis(methacryloyloxymethyl)tricyclo[5.2.1.0/2,6]decane, bis(acryloyloxymethyl)tricyclo[5.2.1.0/2,6]decane, and mixtures thereof. More preferably, the base monomer M2 is bis(acryloyloxymethyl)tricyclo[5.2.1.0/2,6]decane. The base monomer M2 can be a commercially available monomer, such as tricyclo[5.2.1.0/2,6]decanedimethanol diacrylate from PolyScience. However, it may also be monomers obtainable by an esterification reaction, for example according to the production examples of EP0235836B1 or U.S. Pat. No. 4,131,729/DE2816823. Industrially available base monomers M2 based on tricyclo[5.2.1.0/2,6]decanedimethanol di(meth)acrylate generally comprise isomer mixtures in which the exocyclic methylene groups are attached to different backbone carbon atoms, depending on the isomer.

The monomer mixture may comprise a base monomer M3 that differs from the base monomers M1 of the formula 1 and M2 of the formula 2.

The base monomer M3 is preferably selected from urethane-based monomers.

Suitable base monomers M3 may be selected from difunctional urethane (meth)acrylates, polyfunctional urethane (meth)acrylates, and mixtures thereof.

The base monomer M3 is preferably urethane di(meth)acrylates. Urethane di(meth)acrylates are preferably selected from linear or branched alkylene bis(urethane(meth)acrylates) and urethane di(meth)acrylate-functionalized polyethers.

Preference is given to difunctional urethane (meth)acrylates selected from difunctional urethane(meth)acrylates having a divalent alkylene group and ones having a divalent cyclic aliphatic hydrocarbon group.

Difunctional urethane (meth)acrylates having a divalent alkylene group are preferably selected from linear or branched urethane di(meth)acrylates functionalized with a divalent alkylene group, such as bis(methacryloyloxy-2-ethoxycarbonylamino)alkylene.

The difunctional urethane (meth)acrylates may thus be compounds of the formula 14 below, having a group B selected from a divalent linear or branched alkylene group and a divalent cyclic aliphatic hydrocarbon group, having a group Z selected from linear and branched C2-C8 hydrocarbon radicals in which one or more carbon atoms may optionally be replaced by oxygen, nitrogen or sulfur, and having groups X that may each independently be methyl or H. An example is bis(methacryloyloxy-2-ethoxycarbonylamino)alkylene. The divalent alkylene comprises preferably 2,2,4-trimethylhexamethylene and/or 2,4,4-trimethylhexamethylene. Preference is given to 1,6-bis(methacryloyloxy-2-ethoxycarbonylamino)-2,4,4-trimethylhexane. Examples include UDMA and HEMA-TDMI.

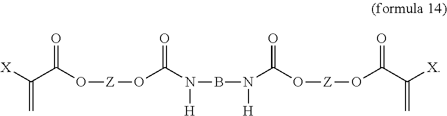

The base monomer M3 may also be urethane di(meth)acrylate-functionalized polyethers having alkylene group(s), such as bis(methacryloyloxy-2-ethoxycarbonylamino)-substituted polyalkylene ethers. Group B from formula 14 is in these cases a polyether group. Preference is given to compounds containing bis(methacryloyloxy-2-ethoxycarbonylamino) that include linear or branched alkylene groups having 3 to 20, preferably 3 to 9, carbon atoms or divalent cyclic aliphatic groups having 3 to 20, preferably 3 to 9, carbon atoms. This may also be an alkylene substituted with methyl groups or a cyclohexyl group substituted with methyl groups.

In addition, the base monomer M3 may also be HP-UDMA, a reaction product of 3-hydroxypropyl methacrylate and trimethylhexamethylene diisocyanate, or HP-UDA, a reaction product of 3-hydroxypropyl acrylate and trimethylhexamethylene diisocyanate.

Urethane(meth)acrylates with divalent cyclic aliphatic hydrocarbon group are obtainable by reacting 2 mol of 2-hydroxyethyl methacrylate (HEMA) or 2 mol of 2-hydroxyethyl acrylate (HEA) with 1 mol of cyclic aliphatic diisocyanate. Suitable diisocyanates are isophorone diisocyanate (1-isocyanato-3-isocyanatomethyl-3,5,5-trimethylcyclohexane) or H12-MDI (1-isocyanato-4-[(4-isocyanatocyclohexyl)methyl]cyclohexane and other cyclic diisocyanates. Examples include UDA-IPDI, the reaction product of 2 molecules of 2-hydroxyethyl acrylate (HEA) and one molecule of isophorone diisocyanate (IPDI), and also UDMA-IPDI, the adduct of 2 molecules of 2-hydroxyethyl methacrylate (HEMA) and one molecule of isophorone diisocyanate.

Suitable base monomers M3 are available for example under the following trade or brand names: Ebecryl 230 (aliphatic urethane diacrylate), CN9200 (aliphatic urethane diacrylate), Ebecryl 210 (aromatic urethane diacrylate oligomers), Ebecryl 270 (aliphatic urethane diacrylate oligomer), Photomer 6210 (aliphatic urethane diacrylate), Photomer 6891 (aliphatic urethane diacrylate), UDMA, Genomer 4256 (aliphatic urethane dimethacrylate, Genomer 4267 (aliphatic urethane diacrylate), Genomer 4259 (aliphatic urethane diacrylate), RCX 18-059 (aliphatic urethane diacrylate), CN 1963CG (aliphatic urethane methacrylate), CN 1993CG (aliphatic urethane methacrylate), PRO 21252 (aliphatic urethane acrylate), H1391 (hydroxypropylurethane dimethacrylate), X851-1066 (urethane dimethacrylate), X726-000 (PEG 400 extended urethane dimethacrylate), and urethane methacrylate 14-774 (aliphatic urethane dimethacrylate), Genomer 4277 (aliphatic urethane dimethacrylate).

The base monomer M3 is preferably selected from 7,7,9-(or 7,9,9-)trimethyl-4,13-dioxo-3,14-dioxa-5,12-diazahexadecane-1,16-diyl-bis(2-methylacrylate) (UDMA), 7,7,9-(or 7,9,9-)trimethyl-4,13-dioxo-3,14-dioxa-5,12-diazahexadecane-1,16-diol diacrylate (UDA), the reaction product of two molecules of 2-hydroxyethyl acrylate (HEA) and one molecule of isophorone diisocyanate (IPDI) (UDA-IPDI), and mixtures thereof.

In a preferred embodiment, the base monomer M3 is selected from UDMA, UDA, UDA-IPDI, and mixtures thereof.

In addition, the monomer mixture may contain other monomers. Other monofunctional monomers are preferably selected from MMA (methyl methacrylate), EMA (ethyl methacrylate), n-BMA (nbutyl methacrylate), IBMA (isobutyl methacrylate), t-BMA (tert-butyl methacrylate), EHMA (2-ethylhexyl methacrylate), LMA (lauryl methacrylate), TDMA (tridecyl methacrylate), SMA (stearyl methacrylate), CHMA (cyclohexyl methacrylate), BZMA (benzyl methacrylate), IBXMA (isobornyl methacrylate), MAA (methacrylic acid), HEMA (2-hydroxyethyl methacrylate), HPMA (2-hydroxypropyl methacrylate), DMMA (dimethylaminoethyl methacrylate), DEMA (diethylaminoethyl methacrylate), GMA (glycidyl methacrylate), THEMA (tetrahydrofurfuryl methacrylate), AMA (allyl methacrylate), ETMA (ethoxyethyl methacrylate), 3FMA (trifluoroethyl methacrylate), 8FMA (octafluoropentyl methacrylate), IBA (isobutyl acrylate), TBA (tert-butyl acrylate), LA (lauryl acrylate), CEA (cetyl acrylate), STA (stearyl acrylate), CHA (cyclohexyl acrylate), BZA (benzyl acrylate), IBXA (isobornyl acrylate), 2-MTA (2-methoxyethyl acrylate), ETA (2-ethoxyethyl acrylate), EETA (ethoxyethoxyethyl acrylate), PEA (2-phenoxyethyl acrylate), THFA (tetrahydrofurfuryl acrylate), HEA (2-hydroxyethyl acrylate), HPA (2-hydroxypropyl acrylate), 4HBA (4-hydroxybutyl acrylate), DMA (dimethylaminoethyl acrylate), 3FA (trifluoroethyl acrylate), 17FA (heptadecafluorodecyl acrylate), 2-PEA (2-phenoxyethyl acrylate), TBCHA (4-tert-butylcyclohexyl acrylate), EHA (2-ethylhexyl acrylate), 3EGMA (triethylene glycol monomethacrylate), isodecyl methacrylate, isodecyl acrylate, trimethylcyclohexyl methacrylate, trimethylcyclohexyl acrylate, tert-butylcyclohexyl acrylate, SR256 ((2-(2-ethoxyethoxyethyl acrylate), SR257C (C16-C18 alkyl acrylate), CD278 (diethylene glycol monobutyl ether acrylate), SR440 (isooctyl acrylate), SR484 (octyldecyl acrylate), adamantyl methacrylate (CAS=16887-36-8), dicyclopentanyl methacrylate (CAS=34759-34-7), dicyclopentenyloxyethyl methacrylate (CAS=68586-19-6), dicyclopentanyl acrylate (CAS=79637-74-4), dicyclopentenyloxyethyl acrylate (CAS=65983-31-5), and dicyclopentanylmethyl acrylate (CAS=93962-84-6). Other difunctional and polyfunctional monomers are preferably selected from DDDMA (decane-1,10-diol dimethacrylate), DDDA (decane-1,10-diol diacrylate), NDDA (nonane-1,9-diol diacrylate), NDDMA (nonane-1,9-diol dimethacrylate), HDDMA (hexane-1,6-diol dimethacrylate), HDDA (hexane-1,6-diol diacrylate), PDDMA (pentane-1,5-diol dimethacrylate), PDDA (pentane-1,5-diol diacrylate), BDDMA (butane-1,4-diol dimethacrylate), BDDA (butane-1,4-diol diacrylate), PRDMA (propane-1,3-diol dimethacrylate), PRDA (propane-1,3-diol acrylate), GDMA (glycerol dimethacrylate), PEG400DA (polyethylene glycol 400 diacrylate), PEG400DMA (polyethylene glycol 400 dimethacrylate), PEG300DA (polyethylene glycol 300 diacrylate), PEG300DMA (polyethylene glycol 300 dimethacrylate), PEG200DA (polyethylene glycol 200 diacrylate), PEG600DA (polyethylene glycol 600 diacrylate), NPG(PO)2DA (propoxylated (2) neopentyl glycol diacrylate), NPG(PO)2DMA (propoxylated (2) neopentyl glycol dimethacrylate), EGDMA (ethylene glycol dimethacrylate), EGDA (ethylene glycol diacrylate), TEGDMA (triethylene glycol dimethacrylate), TEDA (triethylene glycol diacrylate), 4EGDMA (tetraethylene glycol dimethacrylate), 4EGDA (tetraethylene glycol diacrylate), BGDMA (1,3-butylene glycol dimethacrylate), BGDA (1,3-butylene glycol diacrylate), DEGDMA (diethylene glycol dimethacrylate), DEGDA (diethylene glycol diacrylate), NPG-DMA (neopentyl glycol dimethacrylate), NPG-DA (neopentyl glycol diacrylate), TPGDMA (tripropylene glycol dimethacrylate), TPGDA (tripropylene glycol diacrylate), SR341 (3-methylpentane-1,5-diol diacrylate), CD536 (dioxane glycol diacrylate), TMPTMA (trimethylolpropane trimethacrylate), TMPTA, trimethylolpropane triacrylate, DTMPTMA (di-trimethylolpropane tetramethacrylate); DTMPTA (di-trimethylolpropane tetraacrylate); DiPENTMA (di-pentaerythritol pentamethacrylate); DiPENTA (di-pentaerythritol pentaacrylate), DPEHMA (di-pentaerythritol hexamethacrylate), DPEHA (di-pentaerythritol hexaacrylate), Miramer M340 (pentaerythritol triacrylate), SR494 (ethoxylated pentaerythritol tetraacrylate), Miramer M4004 (pentaerythritol n-EO tetraacrylate), SR593 (ethoxylated pentaerythritol triacrylate), ethoxylated trimethylolpropane trimethacrylate, ethoxylated trimethylolpropane triacrylate, propoxylated trimethylolpropane trimethacrylate, propoxylated trimethylolpropane triacrylate, ethoxylated pentaerythritol trimethacrylate, ethoxylated pentaerythritol triacrylate, ethoxylated pentaerythritol tetramethacrylate, ethoxylated pentaerythritol tetraacrylate, ethoxylated di-pentaerythritol trimethacrylate, ethoxylated di-pentaerythritol triacrylate, ethoxylated di-pentaerythritol tetramethacrylate, ethoxylated di-pentaerythritol tetraacrylate, ethoxylated di-pentaerythritol pentamethacrylate, ethoxylated di-pentaerythritol pentaacrylate, ethoxylated di-pentaerythritol hexamethacrylate, ethoxylated di-pentaerythritol hexaacrylate, propoxylated pentaerythritol trimethacrylate, propoxylated pentaerythritol triacrylate, propoxylated pentaerythritol tetramethacrylate, propoxylated pentaerythritol tetraacrylate, propoxylated di-pentaerythritol trimethacrylate, propoxylated di-pentaerythritol triacrylate, propoxylated di-pentaerythritol-tetramethacrylate, propoxylated di-pentaerythritol tetraacrylate, propoxylated di-pentaerythritol pentamethacrylate, propoxylated di-pentaerythritol pentaacrylate, propoxylated di-pentaerythritol hexamethacrylate, propoxylated di-pentaerythritol hexaacrylate, Miramer M320 (propoxylated glycerol triacrylate), SR 9019 (propoxylated glycerol triacrylate), SR 9020 (propoxylated glycerol triacrylate), SR 9021 (highly propoxylated glycerol triacrylate), Genomer 3364 (modified acrylated polyether polyol), SR 9041 (pentaacrylate esters). The other mono-, di-, and polyfunctional monomers may be present alone or in a mixture.

Preferred other monomers are triethylene glycol dimethacrylate (TEGDMA), tripropylene glycol diacrylate (TPGDA), 2-hydroxyethyl acrylate (HEA), dicyclopentanylmethyl acrylate (TCDA), and mixtures thereof.

It is preferable that one or more of the following base monomers are present in the following mass fractions based on the total mass of the monomer mixture:
base monomer M1 from 2% to 75% by weight, preferably from 5% to 68% by weight, more preferably from 13% to 63% by weight, even more preferably from 15% to 52% by weight;
base monomer M2 from 5% to 96% by weight, preferably from 12% to 65% by weight, more preferably from 30% to 63.5% by weight, even more preferably from 30% to 52% by weight;
base monomer M3 from 0% to 75% by weight, preferably from 0.1% to 65% by weight, more preferably from 12% to 65% by weight, even more preferably from 12% to 64% by weight.

In addition, the other monomers may be present in mass fractions of 0-15% by weight, preferably 0.1-15% by weight, more preferably 1-10% by weight, more preferably 1-4% by weight, even more preferably 1-2% by weight, based on the total mass of the monomer mixture.

It is preferable that the monomer mixture comprises the base monomers M1 and M2 in a mass fraction of 25% by weight or more, more preferably of 30% by weight or more, even more preferably of 40% by weight or more, further preferably of 50% by weight or more, even further preferably of 60% by weight or more, even further preferably of 70% by weight or more, even further preferably of 80% by weight or more, even further preferably of 90% by weight or more, based on the total masses of the monomer mixture, or consists thereof.

According to the invention, it is preferable that a mass ratio Y=m(M2)/m(M1) of the base monomers M2 to M1 is 0.5≤Y≤20, preferably 0.6≤Y≤10, more preferably 0.95≤Y≤5.

The monomer mixture may comprise the base monomers M1, M2, and M3 in a mass fraction of from 80% to 100% by weight, preferably from 85% to 100% by weight, more preferably from 87% to 100% by weight, even more preferably 100% by weight, based on the total mass of the monomer mixture, or consist thereof.

According to the invention, the monomer mixture preferably does not contain any monomer having a bisphenol A structure.

In particular, 2,2-bis[4-(2-hydroxy-3-(meth)acryloyloxypropoxy)phenyl]propane (bis-GMA) and/or ethoxylated bisphenol A di(meth)acrylate (bis-EMA) are absent. The same applies to the polymerizable dental material.

In one embodiment, the monomer mixture preferably does not contain any monomer selected from low-molecular-weight and low-viscosity mono- and di(meth)acrylates. In particular, it does not contain any monomer having a viscosity at a temperature of 23° C. of less than 0.05 Pa·s and/or having partial solubility in water. In particular, the monomer mixture does not contain any di(meth)acrylates having an oligo[ethylenoxy] group or a linear or branched C1-C10 alkylene group. The monomer mixture is more preferably free of hexanediol diacrylate (HDDA), hexanediol dimethacrylate (HDDMA), triethylene glycol diacrylate (TEGDA) and/or triethylene glycol dimethacrylate (TEGDMA). The same applies to the polymerizable dental material.

The viscosity of monomers or of organic resins is specified regularly by the manufacturer and can be determined using a viscometer (e.g. Kinexus Pro from Malvern Instruments Ltd.). A plate-plate geometry with an upper plate diameter of 25 mm and a gap width of 0.1 mm was used. The measurement covered a shear stress range of 0.1 Pa to 50 Pa. The value at 50 Pa shear stress was used for the evaluation. The measurement is performed at a temperature of 23° C., which was monitored and kept constant by the internal temperature control of the instrument.

At a temperature of 23° C., the monomer mixture of the invention preferably has a viscosity of 0.2 to 10 Pa·s, more preferably 1 to 6 Pa·s.

The invention has the advantage that the monomer mixture according to the invention and consequently also the polymerizable dental material according to the invention overcome the disadvantages mentioned above of the prior art. The monomer mixture and the polymerizable dental material can be produced from base monomers that are readily obtainable and that in addition have a reduced toxicity potential. The use of the monomer mixture of the invention for the production of a dental material results in reduced polymerization shrinkage in tandem with good mechanical properties in the dental material obtained. In particular, the monomer mixture can be used to obtain dental materials, in particular dental composites, having improved volume shrinkage and improved flexural strength, and also a good and a good elastic modulus. This is a surprise, given the molecular sizes and structures of the base monomer M1, since an associated reduction in crosslinking density and flexural strength would have been expected.

The monomer mixture and the polymerizable dental material thus preferably do not contain any monomers or other compounds having bisphenol-A-derived structural elements or any low-molecular-weight mono- and di(meth)acrylates having partial solubility in water, in particular no TEGDMA.

The invention further provides a monomer mixture for producing a dental material, comprising:
a. at least two or more base monomers M1 represented by the following empirical formula 1:

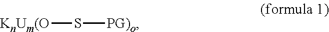

where
   PG=is a polymerizable group selected from —OOC—CH═CH₂ and —OOC—C(CH₃)═CH₂;
   S=is a spacer group selected from unbranched and branched alkylene having 1-10 carbon atoms, which may additionally contain oxygen and/or —OOC— in the carbon chain, and is preferably ethylene;
   o=is oxygen;
   U=is a group represented by the following formula 2:

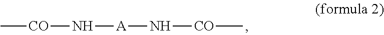

where
   A=is a group selected from a divalent aromatic or aliphatic C6-C20 hydrocarbon group, preferably a divalent C6-C13 aliphatic hydrocarbon group, represented by the following formula 6:

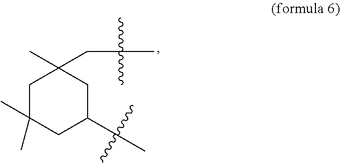 [SMILES: *CC1(C)CC(*)CC(C)(C)C1]

where the two indicated bonds (i.e. the broken lines) each represent the points of attachment to the nitrogen atoms in formula 2;
   K=is a group represented by the following formula 3:

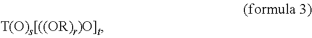

where
   T=is a trivalent hydrocarbon group having 3-7 carbon atoms, preferably a trivalent hydrocarbon group, represented by the following formula 5:

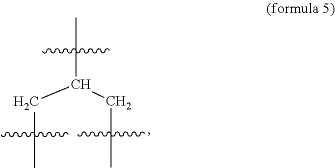 [SMILES: *CC(*)C*]

where the three indicated bonds (i.e. the broken lines) each represent the points of attachment to the oxygen atoms in formula 3,
   O=is oxygen,
   R=is in each case independently selected from an ethylene group, a 1,2-propylene group, a 1,3-propylene group and a mixture thereof, preferably a 1,2-propylene group,
   r=is in each case independently 1-12, preferably 1-9, more preferably 1-6,
   s=is 0 or 1, preferably 0,
   t=is 2 or 3, preferably 3,
   where the condition must be met that s+t=3;
   n=1-9, preferably 1-7, more preferably 1-5;
   where the condition must be met that m=2n+1 and o=n+2;
b. optionally at least one base monomer M2 of the following formula 4:

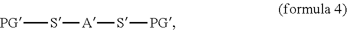

where
   PG′=is a polymerizable group selected from —OOC—CH═CH₂ and —OOC—C(CH₃)═CH₂;
   S′=is a spacer group selected from unbranched and branched alkylene having 1-10 carbon atoms, which may additionally contain oxygen and/or —OOC— in the carbon chain, and is preferably methylene;
   or S′ is absent;
   A′=is an aliphatic polycyclic group, preferably an aliphatic tricyclic hydrocarbon group, in which one or more hydrogen atoms may each be independently replaced by C1-C4 alkyl radicals, C1-C4 alkoxy radicals, fluorine atoms, chlorine atoms or trifluoromethyl groups, more preferably is tricyclodecanylene, even more preferably tricyclo[5.2.1.0/2,6]decanylene.

The monomer mixture preferably comprises a plurality of base monomers M1, more preferably more than two base monomers M1, further preferably more than three base monomers M1, even further preferably more than four base monomers M1, even further preferably more than five base monomers M1, and so on. It is preferably a mixture that, in addition to base monomers, also includes base oligomers. In accordance with the invention, such a mixture is also referred to as a mixture of base monomers M1.

It is preferable that, in addition to the base monomer M1 where n=1 (i.e. a monomer), at least one base monomer M1 where n is greater than 1 (i.e. an oligomer) is also present in the mixture. The mass fraction of the base monomer(s) M1 where n is greater than 1, which can be determined through fractionation by gel-permeation chromatography using a refractive index detector (measurement with visible light), is preferably 5-70% by weight, more preferably 10-60% by weight, even more preferably 15-50% by weight, based on the total mass of all base monomers M1 of a monomer/oligomer series of n=1-9.

The distribution of the base monomers M1 in respect of n can vary within wide ranges. It may be the case that the base monomers M1 in which n=2-5 or n=2-4 have the highest mass fraction based on the total mass fraction of the base monomers M1. However, it may also be the case that the base monomer(s) M1 where n=1 have the highest mass fraction compared to each individual base monomer M1 present in the monomer mixture where n=2-9.

The base monomers M1 where n>1 are present as base monomers where n=2-9, preferably where n=2-7, more preferably where n=2-5.

In one embodiment, all base monomers M1 of a monomer/oligomeric series where n=1-9, preferably where n=1-7, more preferably where n=1-5, may be present side by side. This means that if at least one compound in each case is present for each n from 1-9, then at least 9 compounds (i.e. a monomer for n=1 and eight oligomers for n=2, 3, 4, etc.) would be present in the monomer mixture (or monomer/oligomer mixture). For n=1-7 there would then be at least 7 compounds (i.e. a monomer and six oligomers) and for n=1-5 there would be at least 5 compounds (i.e. a monomer and four oligomers) present in the monomer mixture.

Furthermore, for the monomer mixture for producing a dental material where said mixture comprises at least two or more base monomers M1, the same characteristics and conditions essentially apply as for the monomer mixture of the invention for producing a dental material where said mixture comprises at least one base monomer M1.

The invention further provides for the use of the monomer mixture of the invention, preferably as claimed in any of claims 1 to 10, for the production of a polymerizable dental material, preferably a dental composite, core build-up material, root-canal filling material, filling material, underfill material, fixation material, crown material, bridge material, restoration material and/or prosthesis material.

In a preferred embodiment, it is a radically polymerizable dental material.

The invention also provides a polymerizable dental material comprising:
a) the monomer mixture of the invention, preferably as claimed in any of claims 1 to 10;
b) optionally at least one initiator or initiator system for the polymerization;
c) optionally fillers;
d) optionally customary dental additives.

The polymerizable dental material may take the form of a kit. The kit may comprise a single component or a plurality thereof. In the case of a multicomponent kit or system, the production of the dental material takes place immediately before application of the dental material, by mixing the components in the specified mixing ratio and then curing.

### B) Initiator(s)

Suitable initiators or initiator systems are capable of starting polymerization reactions, preferably free-radical polymerization reactions. Such initiators and initiator systems are known to those skilled in the art.

Initiator systems consist of at least one initiator and at least one further compound, such as a co-initiator. These may be distributed over different components of the polymerizable dental material. The dental material according to the invention can be cured thermally, chemically, or photochemically, i.e. by irradiation with UV and/or visible light.

Suitable initiators may be, for example, photoinitiators. These are characterized in that they bring about curing of the material by absorbing light in the wavelength range from 300 nm to 700 nm, preferably from 350 nm to 600 nm and more preferably from 380 nm to 500 nm, and optionally through additional reaction with one or more co-initiators. Preference is given here to phosphine oxides, acylphosphine oxides, bisacylphosphine oxides and derivatives thereof, acyl germananes, as described for example in EP2649981A1, WO2017/055209A1, and EP3153150A1, benzoin ethers, benzil ketals, acetophenones, benzophenones, thioxanthones, bisimidazoles, metallocenes, fluorones, α-dicarbonyl compounds, aryldiazonium salts, arylsulfonium salts, aryliodonium salts, ferrocenium salts, phenylphosphonium salts or a mixture of said compounds.

Particular preference is given to diphenyl(2,4,6-trimethylbenzoyl)phosphine oxide, phenylbis(2,4,6-trimethylbenzoyl)phosphine oxide, benzoin, benzoin alkyl ethers, benzil dialkyl ketals, α-hydroxyacetophenone, dialkoxyacetophenones, α-aminoacetophenones, isopropylthioxanthone, camphorquinone, phenylpropanedione, 5,7-diiodo-3-butoxy-6-fluorone, (η⁶-cumene) (η⁵-cyclopentadienyl) iron hexafluorophosphate, (η⁶-cumene) (η⁵-cyclopentadienyl) iron tetrafluoroborate, (η⁶-cumene) (η⁵-cyclopentadienyl) iron hexafluoroantimonate, substituted diaryliodonium salts, triaryl sulfonium salts or a mixture of said compounds.

Co-initiators used for photochemical curing are preferably tertiary amines, borates, organic phosphites, diaryliodonium compounds, thioxanthones, xanthene, fluorene, fluorone, α-dicarbonyl compounds, dicarbonyl systems as described in WO2021/048313A1, condensed polyaromatics or a mixture of said compounds. Particular preference is given to N, N-dimethyl-ptoluidine, N, N-dialkyl alkylanilines, N, N-dihydroxyethyl-ptoluidine, 2-ethylhexyl p-dimethylaminobenzoate, ethyl p-dimethylaminobenzoate, butyrylcholine triphenylbutylborate or a mixture of said compounds.

It is also possible to use as initiators what are known as thermal initiators, which are able to bring about curing of the material by absorbing thermal energy at elevated temperatures. Preference is given here to using inorganic and/or organic peroxides, inorganic and/or organic hydroperoxides, diethyl α,α′-azobisisobutyrate, α,α′-azobisisobutyronitrile, benzopinacols or a mixture of said compounds. Particular preference is given to diacyl peroxides such as benzoyl peroxide or lauroyl peroxide, cumene hydroperoxide, benzopinacol, 2,2′-dimethylbenzopinacol or a mixture of said compounds.

For chemical curing at room temperature, a redox initiator system is generally used that consists of one or more initiators and one or more co-initiators serving as activator. For storage stability reasons, individual components of an initiator system are incorporated in spatially separated components of the dental material according to the invention, i.e. a multicomponent, preferably two-component, material is present. As initiator(s), preference is given to using inorganic and/or organic peroxides, inorganic and/or organic hydroperoxides, barbituric acid derivatives, malonyl sulfamides, protic acids, Lewis or Brønsted acids or compounds that release such acids, carbenium ion donors such as methyl triflate or triethyl perchlorate or a mixture of said compounds, and as co-initiator(s) preference is given to using tertiary amines, heavy metal compounds, especially compounds of the 8th and 9th group of the periodic table (“iron group and copper group”), compounds containing ionogenically bound halogens or pseudohalogens, for example quaternary ammonium halides, weak Brønsted acids, for example alcohols and water, or a mixture of said compounds.

The dental material according to the invention may also comprise any conceivable combination of the initiators and co-initiators described above. An example is what are known as dual-curing dental materials, which comprise both photoinitiators and optionally the corresponding co-initiators for photochemical curing and initiators and corresponding co-initiators for chemical curing at room temperature.

The polymerizable dental material according to the invention is preferably light-curing. In a preferred embodiment, camphorquinone (CQ) is present as initiator and 2-ethylhexyl p-dimethylaminobenzoate (EHA) or ethyl p-dimethylaminobenzoate is present as co-initiator.

### C) Fillers

The polymerizable dental material according to the invention may comprise further customary dental additives. The filler particles are not defined as having a particular particle shape. Rather, fillers having a spherical, flake-like, platelike, needle-like, leaf-like or irregular shape may be very readily used. The filler particles preferably have an average particle diameter of from 5 nm to 100 μm, more preferably from 5 nm to 50 μm.

Suitable fillers may be selected from a wide variety of materials commonly used in dental products. Through the selection of the filler it is possible to adjust for example the fluidity, viscosity, consistency, color tone, radiopacity, and mechanical stability of the dental material. The fillers can be broadly divided according to their chemical nature into three different classes: inorganic fillers, organic fillers, and organic-inorganic composite fillers. The fillers can be used not just individually, but also in combination with one other.

Inorganic fillers used may be ground powders of natural or synthetic glasses or crystalline inorganic substances in various sizes and states (monodisperse, polydisperse). Suitable materials include quartz, cristobalite, glass ceramics, feldspar, barium silicate glasses (for example those available under the Kimble Ray-Sorb T3000, Schott 8235, Schott GM27884, Schott G018-053, and Schott GM39923 trade names), barium fluorosilicate glasses, strontium silicate glasses, strontium borosilicate glasses (for example those available under the Ray-Sorb T4000, Schott G018-093, Schott G018-163, and Schott GM32087 trade names), lithium aluminosilicate glasses, barium glasses, calcium silicates, sodium aluminosilicates, fluoroaluminosilicate glasses (for example those available under the Schott G018-091 and Schott G018-117 trade names), zirconium or cesium boroaluminosilicate glasses (for example those available under the Schott G018-307, G018-308 and G018-310 trade names), zeolites, and apatites. The fillers preferably have a median particle size d50 of 0.01-15 μm, preferably a median particle size d50 of 0.2-5 μm, and more preferably a median particle size of 0.2-1.5 μm. It may be preferable that the median particle size d50 is between 0.1-0.5 μm. In such cases, it is particularly preferable that the median particle size d90 is less than 1.0 μm. In addition, discrete, non-agglomerated, non-aggregated, organically surface-modified nanoparticles may be used to achieve a more uniform filling of the dental material and to increase the hardness and abrasion resistance.

Nanoparticles are understood in this context as meaning spherical particles having a median particle size of less than 200 nm. The median particle size is preferably less than 100 nm and more preferably less than 60 nm. The smaller the nanoparticles, the better they are able to fulfill their function of filling the cavities between the coarser particles. The materials for the nanoparticles are by preference oxides or mixed oxides and preferably selected from the group consisting of oxides and mixed oxides of the elements silicon, titanium, yttrium, strontium, barium, zirconium, hafnium, niobium, tantalum, tungsten, bismuth, molybdenum, tin, zinc, ytterbium, lanthanum, cerium, aluminum, and mixtures thereof. Preferred oxidic nanoparticles are not agglomerated. In order to permit good incorporation of the nanoparticles into the polymer matrix of a composite material, the surfaces of the nanoparticles are organically modified. The surface treatment of the fillers is preferably carried out using a silanizing agent. A particularly suitable adhesion promoter is methacryloyloxypropyltrimethoxysilane. Commercially available nanoscale, non-agglomerated and non-aggregated silica sols that can be used are marketed for example under the “Nalco Colloidal Silicas” (Nalco Chemical Co.), “Ludox colloidal silica” (Grace) or “Highlink OG” (Clariant) names.

Submicron fillers or microfillers consisting of agglomerated nanoscale particles may likewise be used, particularly if their specific surface area (determined by the Brunauer, Emmet, and Teller method) is in the range between 100 to 400 m²/g. Fumed silica or wet-precipitated silica are preferred. Suitable non-surface-treated silicon dioxide filler products that can be used are commercially available under the Aerosil™ (“OX50”, “90”, “130”, “150”, “200”, “300”, “380”, and “R8200” from Evonik Industries AG, Essen, Germany), Cab-O-Sil (“LM-150”, “M-5”, “H5”, “EH-5” from Cabot Corp., Tuscola, IL), HDK™ (“S13”, “V15”, “N20”, “T30”, “T40”, Wacker-Chemie AG, Munich, Germany) and Orisil™ (“200”, “300”, “380”, Orisil, Lviv, Ukraine) names.

Particularly advantageous abrasion resistance and gloss resistance properties can be achieved in the dental material by using aggregated nanoscale particles based on mixed oxides of silicon dioxide and zirconium dioxide. A suitable filler can be produced by a process described for example in U.S. Pat. No. 6,730,156 (example A). The filler thus produced can then be surface-treated according to a method such as that described in U.S. Pat. No. 6,730,156 (for example production example B).

The aggregated fillers preferably have a median secondary particle size of 1-15 μm, preferably a median secondary particle size of 1-10 μm, and more preferably a median secondary particle size of 2-5 μm.

In order to achieve high filler contents in tandem with good esthetics and abrasion stability, it can be particularly advantageous to use spherical submicroparticles based on silicon-zirconium mixed oxides, such as those described in DE 19524362 A1 or US2020/0121564 A1.

Appreciable amounts of selected radiopaque fillers may additionally be present. The addition of radiopaque particles to the dental material is advantageous, since this makes it possible to distinguish between healthy dental hard substance and the restoration. Suitable radiopaque fillers comprise particles of metal oxides, metal fluorides or barium sulfate. Oxides and fluorides of heavy metals of atomic number greater than 28 are preferred. The metal oxides and fluorides should be selected so as to affect the color of the restoration as little as possible. Metal oxides and metal fluorides of atomic number greater than 30 are more suitable. Suitable metal oxides are oxides of yttrium, strontium, barium, zirconium, hafnium, niobium, tantalum, tungsten, bismuth, molybdenum, tin, zinc, lanthanides (elements having an atomic number of 57 to 71), cerium, and combinations thereof. Suitable metal fluorides are for example yttrium trifluoride and ytterbium trifluoride. Suitable with particular preference here are irregularly shaped or spherical YbF₃ or YF₃ particles having an average primary particle grain size of from 40 nm to 1.5 μm and more preferably core-shell combination products having a YF₃ or YbF₃ core and SiO shell, very particularly preferably where the surface of the Sio shell is silanized. In particular, such a core-shell combination product has a refractive index of 1.48 to 1.54 and a measured median grain size of the agglomerated particles of between 0.5 and 5 μm.

Examples of suitable organic fillers are filled and unfilled, powdered polymers or copolymers based on polymethyl methacrylate (PMMA), polyethylene methacrylate, polypropylene methacrylate, polybutyl methacrylate, (PBMA), polyvinyl acetates (PVAc), polyethylene glycol (PEG), polypropylene glycol (PPG), polyvinyl alcohol (PVA), polyurethanes (PU), polyurea, methyl methacrylate-ethyl methacrylate copolymer, ethylene-vinyl acetate copolymer, and styrene-butadiene copolymer. In addition, the organic filler may comprise a biologically active component, a specific pigment, a polymerization initiator, a stabilizer or similar that had been added during the production process. The organic fillers may be used alone or as mixtures.

Advantageous polishing properties in tandem with higher filler contents can be achieved in the dental materials when what are known as organic-inorganic composite fillers are used. These fillers can be produced by processing a polymerizable monomer with an inorganic filler into a paste, then curing this by polymerization and then finely grinding prior to use as a filler. Preference is given here to using microfillers as inorganic filler. After grinding, the fillers preferably have a median particle size of 0.05-100 μm, preferably a median particle size of 0.5-50 μm, and more preferably a median particle size of 1-30 μm.

It is preferable that the fillers in the dental materials are surface-modified. This is done for example by subjecting the described inorganic or organic-inorganic composite fillers, prior to use, to a surface treatment in order to improve the compatibility, affinity, and the incorporation of the fillers into the resin mixture. This treatment organically modifies the surfaces of the inorganic particles, i.e. the surfaces have organic structural elements. All methods known to those skilled in the art can be employed here. Silanizing agents are preferred for inorganic fillers bearing surface OH groups. Examples here include γ-methacryloyloxyalkyltrimethoxysilanes (number of carbon atoms between the methacryloyloxy group and the silicon atom: 3 to 12), γ-methacryloyloxyalkyltriethoxysilanes (number of carbon atoms between the methacryloyloxy group and the silicon atom: 3 to 12) or silicone compounds such as vinyltrimethoxysilane, vinylethoxysilane, and vinyltriacetoxysilane. The silanizing agent is particularly preferably methacryloyloxypropyltrimethoxysilane.

Inorganic fillers that have few surface OH groups or none at all are preferably used with different surface modifiers, for example surface-treated with titanates, aluminates, zircoaluminates, surfactants, fatty acids, organic acids, inorganic acids or metal alkoxides. Surface modification agents for salts of barium, strontium, and rare earth metals are particularly preferably organic compounds bearing N-, P-, S- and/or O-containing functional groups (for example polyols, sulfoxides, phosphinic esters, phosphonic esters, trialkylphosphines, carboxylic acids, and carboxylic esters). Particularly suitable here is 10-methacryloyloxydecyl dihydrogen phosphate.

Particularly in the case of agglomerated silicon-dioxide-based nanofillers, the surface modifications may consist of groups reactive to free radicals, such as the abovementioned methacryloyloxyalkyl groups, or of groups unreactive to free radicals. Suitable unreactive groups are for example trimethylsilyl, dimethylsilylene or methylsilylidene groups, which can be applied to the surface by silanization with hexamethyldisilazane, dimethyldimethoxysilane or methyltrimethoxysilane, for example. Suitable non-reactive surface-modified agglomerated nanofillers are commercially available under the Aerosil R8200, Aerosil R812S, Aerosil R805, Aerosil R202, and Aerosil R974 (Evonik Industries AG, Essen, Germany) or HDKH2000 and HDKH200/4 (Wacker Chemie, Burghausen, Germany) names. More preferably, the agglomerated nanofillers may be modified with groups that are reactive in free-radical processes, for example methacryloyl groups. A commercial agglomerated nanofiller product modified to be reactive to free radicals is available under the Aerosil R7200 name (Evonik Industries AG, Essen, Germany).

The agglomerated nanofillers may preferably be present in largely deagglomerated form, as described for example in EP1720206.

A dental material according to the invention may contain a proportion of filler particles of between 0% and 95% by weight, preferably from 1% to 95% by weight, based on the total mass of the polymerizable dental material. The amount of the filler fraction is selected in accordance with the indication for the dental product. For instance, for stable, modelable filling composites, for dental compositions for producing inlays, onlays or overlays, and for compositions for producing dental CAD-CAM materials to be subtractively processed, the highest possible amounts of fillers are used. These compositions generally have filler contents of from 75% by weight to 92% by weight based on the total composition. Flowable dental composites, fixation composites, core build-up materials, crown materials, and bridge materials and also dental materials to be processed by stereolithographic methods generally have an average filler content ranging from 40% to 80% by weight based on the total composition, whereas dental varnishes, dental sealing materials, dental infiltrants, lower-viscosity dental materials to be processed by stereolithographic processes, and dental adhesives employ fillers in a content ranging from 18 to 40% by weight based on the total composition. The filler content ranges stated above should be understood as guide values only; departures from this are also possible, depending on the selected filler.

### d) Customary Dental Additives

The polymerizable dental material according to the invention may comprise further customary dental additives. Customary dental additives are known to those skilled in the art; preferred additives are inhibitors, stabilizers, accelerators, dyes, fluoridating agents, remineralizing agents, radiopaques, and film formers.

Inhibitors and stabilizers are employed in particular to prevent premature polymerization. These are substances that react with reactive radicals to form more stable scavenging products. The addition of inhibitors or stabilizers makes it possible to improve the storage stability of compositions that have not yet been cured. Inhibitors can also be employed to adjust the processing time of hardening systems to within a suitable range. Suitable inhibitors are for example phenol derivatives such as hydroquinone monomethyl ether (HOME) or 2,6-di-tert-butyl-4-methylphenol (BHT). Further inhibitors, such as tert-butylhydroxyanisole (BHA), 2,2-diphenyl-1-picrylhydrazyl radicals, galvinoxyl radicals, triphenylmethyl radicals, 2,3,6,6,-tetramethylpiperidinyl-1-oxyl radicals (TEMPO), TEMPO derivatives, and phenothiazine and derivatives of this compound are described in EP 0783880 B1. Alternative inhibitors can be found in DE 10119831 A1 or in EP 1563821 A1.

As stabilizer, the polymerizable dental material may in particular comprise 2,6-di-tert-butyl-4-methylphenol (BHT).

Customary dental additives present in the dental material according to the invention may include UV stabilizers. UV stabilizers are used in particular to stabilize the dental material against degradation or discoloration by UV radiation. Examples of UV absorbers are 2-hydroxy-4-methoxybenzophenone, phenyl salicylate, 3-(2′-hydroxy-5′-methylphenyl)benzotriazole or diethyl 2,5-dihydroxyterephthalate.

Customary dental additives present in the dental material according to the invention may include one or more fluoride-releasing substances in finely divided particulate form. Fluoride-releasing substances may be water-soluble fluorides such as sodium fluoride or amine fluoride. Other suitable fluoride-releasing substances are sparingly soluble fluorides of main group 2. Fluoride-containing glasses are suitable sources of fluoride too.

Other suitable additives are finely particulate substances that release calcium and/or phosphate and accordingly have a remineralizing effect. Suitable remineralizing substances are calcium-phosphate compounds such as hydroxyapatite, brushite, monocalcium phosphate, fluoroapatite, and bioactive glasses such as those mentioned in DE10111449A1, DE102005053954A1 or U.S. Pat. No. 9,517,186B2.

The dental material according to the invention may comprise a colorant or colorant mixture selected from fluorescent dyes, fluorescent pigments, organic color pigments, inorganic color pigments, and mixtures thereof.

A fluorescent colorant or pigment is preferably an organic fluorescent dye or organic fluorescent pigment, in particular a non-polymerizable organic fluorescent colorant optionally comprising esters of aryl carboxylic acids, such as diethyl 2,5-dihydroxyterephthalate, aryl carboxylic acids, coumarin, rhodamine, naphthalimide or derivatives thereof. Examples of inorganic fluorescent pigments include CaAl₄O₇:Mn²⁺(Ba0.98Eu0.02)MgAl₁₀O₁₇, BaMgF₄:Eu²⁺, and Y(1.995)Ce(0.005)SiO₅. As color pigments, the dental material according to the invention may include organic pigments and also inorganic pigments, such as N,N′-bis(3,5-xylyl) perylene-3,4:9,10-bis(dicarboximide), copper phthalocyanine, and titanate pigments, in particular chromium antimony titanates (rutile structure), spinel black, in particular pigments based on iron oxide black (Fe₃O₄) in which iron is partially replaced by chromium and copper or nickel and chromium or manganese, other iron oxide-based pigments, zinc iron chromite brown spinel, ((Zn,Fe)(Fe,Cr)₂O₄) cobalt zinc aluminate blue spinel and/or titanium oxide.

The constituents in the dental material may be present in the following mass fractions based on the total mass of the dental material according to the invention:
the monomer mixture from 5% to 99% by weight, preferably from 10% to 95% by weight, more preferably from 15% to 85% by weight;
the at least one initiator or initiator system for the polymerization from 0% to 5% by weight, preferably from 0.01% to 5% by weight;
the fillers from 0% to 95% by weight, preferably from 1% to 95% by weight, more preferably from 5% to 90% by weight, even more preferably from 15% to 85% by weight;
the customary dental additives from 0% to 5% by weight, preferably from 0.001% to 5% by weight.

The dental material preferably does not contain any compound having a bisphenol A-based structural element.

The invention further provides the dental material according to the invention, preferably as claimed in either of claim 12 or 13, for use in a treatment process as a dental composite, filling material, underfill material, fixation material, core build-up material, root-canal filling material, crown material, bridge material, restoration material and/or prosthesis material.

The invention in addition provides a cured dental material produced from the polymerizable dental material of the invention, in particular as claimed in either of claim 12 or 13.

The invention may in addition provide a method for producing at least one, preferably at least two or more, base monomers M1, the method comprising the following steps:
a) reacting one or more compounds of the following formula 15:

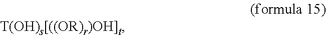

where
   T=is a trivalent hydrocarbon group having 3-7 carbon atoms,
   O=is oxygen,
   R=is in each case independently selected from an ethylene group, a 1,2-propylene group, a 1,3-propylene group and a mixture thereof, preferably a 1,2-propylene group,
   r=is in each case independently 1-12, preferably 1-9, more preferably 1-6,
   s=is 0 or 1, preferably 0,
   t=is 2 or 3, preferably 3,
   where the condition must be met that s+t=3;
   with one or more diisocyanates of the following formula 16:

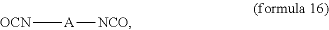

where
   A=is a group selected from a divalent aromatic or aliphatic C6-C20 hydrocarbon group, preferably a divalent C6-C13 aliphatic hydrocarbon group, represented by the following formula 6:

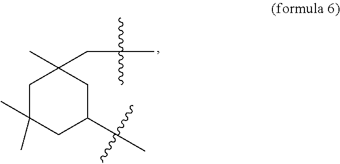 [SMILES: *CC1(C)CC(*)CC(C)(C)C1]

where the two indicated bonds (i.e. the broken lines) each represent the points of attachment to the nitrogen atoms in formula 2;
b) reacting the remaining isocyanate groups of a reaction product from step a) with a compound of the following formula 17:

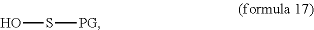

where
   PG=is a polymerizable group selected from —OOC—CH═CH₂ and —OOC—C(CH₃)═CH₂;
   S=is a spacer group selected from unbranched and branched alkylene having 1-10 carbon atoms, which may additionally contain oxygen and/or —OOC— in the carbon chain, and is preferably ethylene.

The reaction in step a) is preferably carried out in a molar ratio of hydroxyl groups x1(OH) to isocyanate groups x₁(NCO) of x₁(NCO)/x₁(OH), subject to the condition that x₁(NCO)/x₁(OH) is >1, more preferably x₁(NCO)/x₁(OH) is between 1.5 and 10, even more preferably x₁(NCO)/x₁(OH) is between 3 and 5.

Preferably, the reaction in step a) is carried out essentially with the formation of urethane groups up to a degree of conversion of at least 95%, more preferably of at least 99%, of all OH groups.

The reaction in step b) is preferably carried out in a molar ratio of hydroxyl groups x₂(OH) to isocyanate groups x₂(NCO) of x₂(OH)/x₂(NCO), subject to the condition that x₂(OH)/x₂(NCO) is ≥1, more preferably x₂(OH)/x₂(NCO) is between 1.0 and 1.4, even more preferably x₂(OH)/x₂(NCO) is between 1.0 and 1.2. Through the choice of the ratio x₁(NCO)/x₁(OH) it possible in particular to control the ratio of the weight fractions of the base monomer M1 where n=1 to the weight fraction of the monomers M1 where n≥2.

The reactions in steps a) and/or b) can take place in a solvent or else without solvent. The solvent may be a low-viscosity resin that does not interfere with the reactions. Suitable low-viscosity resins may be those described as monomer M2 and/or as other monomers, provided they do not react under the reaction conditions employed.

It may be preferable that a reaction product from step b) (i.e. the base monomer M1 or a mixture of base monomers M1) is mixed with the further monomers of the monomer mixture of the invention immediately after completion of the reaction. Further monomers in the context of the invention include the monomers M2 and M3 and also the other monomers. Volatile constituents such as solvents are in that case preferably removed only after the addition of the further monomers. Such a procedure has the advantage that the monomer mixture still has low viscosity and remains stirrable.

However, it may also be preferable that the reaction product from step b) is dried before the addition with the further monomers of the monomer mixture of the invention. This can be advantageous, because the excess proportion of the compound of the formula 17 can be significantly reduced by the drying process. Drying can be effected using a thin-film evaporator, for example. At the end of the drying process, the monomer mixture of the invention can then be produced by adding and mixing with the further monomers.

The invention may in addition provide a monomer mixture for producing a dental material comprising at least one, preferably at least two or more, base monomers M1, that is produced by such a method.

The invention will now be described by way of example with reference to some advantageous embodiments.

### EXAMPLES

For the following examples, the compounds used in the following Table 1 were used in particular.

TABLE 1
Compounds and substances used in the examples.

| Abbreviation | Name |
|---|---|
| OPUA | (Oligo)urethane acrylate (base monomer M1) |
| TCDDA | Bis(acryloyloxymethyl)tricyclo[5.2.1.0/2,6]- |
| | decane [CAS No. 42594-17-2] |
| | (base monomer M2) |
| TCDDMA | Bis(methacryloyloxymethyl)tri- |
| | cyclo[5.2.1.0/2,6]-decane [CAS No. 43048-08-4] |
| | (base monomer M2) |
| UDMA | 7,7,9-(or 7,9,9-)Trimethyl-4,13-dioxo- |
| | 3,14-dioxa-5,12-diazahexadecane-1,16-diol di- |
| | methacrylate [CAS No. 72869-86-4] |
| | (base monomer M3) |
| UDA-IPDI | Reaction product of 2 mol of 2-hydroxyethyl |
| | acrylate with 1 mol of isophorone diisocyanate |
| | [CAS No. 42404-50-2] (base monomer M3) |
| Bis-GMA | Propane-2,2-diylbis[4,1-phenylenoxy(2- |
| | hydroxypropane-3,1-diyl)] bis(2-methylprop- |
| | 2-enoate) [CAS No. 1565-94-2] |
| Exothane 8 | Urethane methacrylates, X891 000, from |
| | Esstech (USA) (base monomer M3) |
| Genomer 4277 | Aliphatic urethane methacrylate, Rahn |
| | (Switzerland) |
| X726 1000 | PEG-extended urethane dimethacrylate, Esstech |
| | (USA) (base monomer M3) |
| MAU | Urethane acrylate methacrylate resin, U-835, |
| | Aldrich (Germany) (base monomer M3) |
| TEGDMA | Triethylene glycol dimethacrylate |
| | [CAS No. 109-16-0] (other monomer OM) |
| TPGDA | Tripropylene glycol diacrylate, |
| | [CAS No. 42978-66-5] (other monomer OM) |
| HEA | 2-Hydroxyethyl acrylate [CAS No. 818-61-1] |
| | (other monomer OM) |
| TCDA | Dicyclopentanylmethyl acrylate [CAS No. |
| | 93962-84-6] (other monomer OM) |
| IPDI | Isophorone diisocyanate [CAS No. 4098-71-9] |
| Glycerol pro- | Propoxylated glycerol having a number-average |
| poxylate 9 | degree of propoxylation of 9; hydroxyl value |
| | 231, CAS No. 25791-96-2 |
| CQ | Camphorquinone |
| EHA | 2-Ethylhexyl p-dimethylaminobenzoate |
| BHT | 2,6-Di-tert-butyl-4-methylphenol |
| Sn-Kat | Dimethyltin dineodecanoate, CAS No. 68928-76-7 |
| Toluene | Toluene, analytical grade, Chemsolute |
| | (Th. Geyer, Germany), cat. No. 752.2500 |
| Toluene, dry | Toluene, dry, (Sigma Aldrich, Germany), |
| | cat. No. 244511-1L. |
| THF, dry | Tetrahydrofuran, dry, stabilized with 250 ppm |
| | of 2,6-di-tert-butyl-4-methylphenol (BHT), |
| | (Sigma Aldrich, Germany), cat. No. 186562-1L. |
| Silica gel | Silica gel 60, 0.063-0.2 mm (Merck, Germany); |
| | cat. No. 1.07734.2500 |

### Example for Oligourethane Acrylate (OPUA)

A preferred example of a base monomer M1 according to the invention is an oligourethane acrylate (OPUA), which is elucidated more particularly below. The oligourethane acrylate (OPUA) according to the invention can be obtainable for example in a mixture with UDA-IPDI (base monomer M3) by reacting a glycerol propoxylate having a number-average degree of propoxylation of 9 per molecule of glycerol with an excess of isophorone diisocyanate and then reacting the excess isocyanate groups with HEA. The monomer mixture thus obtained comprises various OPUAs (as base monomer M1) as well as UDA-IPDI (as base monomer M3) and the excess HEA used (as other monomer). It is also possible to add to the mixture TPGDA (as other monomer), which can serve as thinner resin.

The OPUA thus prepared (as base monomer M1) can be described by the following empirical formula 1:

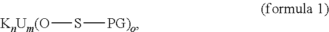

where n=1-4, m=3-9 and o=3-6, where this satisfies the condition that m=2n+1 and o=n+2,
where the proportions of the respective oligomers decrease from n=1 to n=4 and individual structural elements of the OPUA thus prepared look as follows:

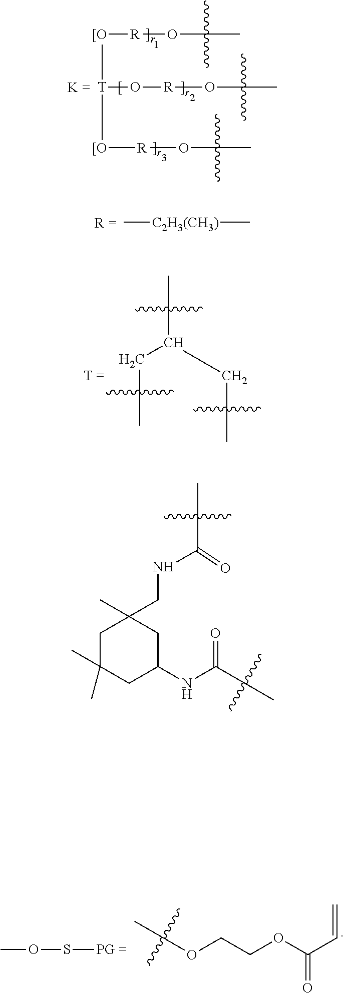

The indicated bonds (i.e. the broken lines) each represent the points of attachment of the groups to the corresponding partner groups. Thus, the three carbon atoms of group T are each attached to the corresponding oxygen atoms of T[((OR)ᵣ₁₋ᵣ₃)O]ₜ of group K. The carbamoyl carbon atoms —NH—CO— of group U may be attached either to an oxygen atom of the —O—S-PG group and to an oxygen atom of group K (T[((OR)ᵣ₁₋ᵣ₃)O]ₜ or to two oxygen atoms of different groups K. The oxygen atom of the —O—S-PG group is always attached to a carbamoyl carbon atom NH—CO— of group U.

The bonding situation is shown below by way of example for a molecule segment T-((OR)ᵣ₂)—O—U—O—S-PG.

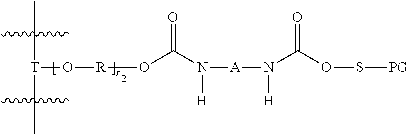

The sum r1+r2+r3 is on average 9.

### Synthesis Example: Preparation of OPUA in a Mixture with UDA-IPDI

Described here is a method by which OPUA can be prepared as base monomer M1 in a mixture with UDA-IPDI.

60 g of glycerol propoxylate 9 is dissolved in 100 ml of toluene and then completely concentrated on a rotary evaporator. A glycerol propoxylate 9 having a residual content of approx. 11.6% by weight of toluene is obtained, which is considered an inert constituent for the purposes of all further syntheses. Weights stated hereinbelow refer to the pure substance glycerol propoxylate 9.

A three-necked flask with dropping funnel and reflux condenser fitted with a drying tube packed with CaCl₂) is initially charged with 15 ml of dry THE with exclusion of moisture. A g of isophorone diisocyanate is added and dissolved with stirring at room temperature. B g of glycerol propoxylate 9 is added at room temperature. 87.2 μl of a 51.5 g/L solution of Sn-Kat in dry toluene is then added with cooling in a water bath and the mixture is stirred for a further 10 min with water cooling. The mixture is then stirred for 3.5 h at a bath temperature of 40° C. C g of HEA is added dropwise and the batch is stirred for a further 0.5 h at 40° C. and then for 62 h at room temperature. No residual isocyanate groups were detectable by IR spectroscopy. The reaction mixture is then applied in a thin layer to an evaporation dish, in which it is initially dried at room temperature in air for 2 days. Residual traces of solvent are removed from the mixture over a period of 2 days at 60° C. in a heating cabinet with forced ventilation. Colorless to pale yellowish, clear viscous substances are obtained in almost quantitative yield.

Batches 1 and 2 shown in Table 3 are obtainable from the mixtures 1 and 2 as per Table 2 below.

TABLE 2
Mixing ratios for the preparation of batches
1 and 2 containing OPUA 3 shown in Table 3.

| | | Weight B | |
|---|---|---|---|
| | Weight A | Glycerol pro- | Weight C |
| | IPDI [g] | poxylate 9 [g] | HEA [g] |
| Mixture for | 4.19 | 2.91 | 3.58 |
| batch 1 | | | |
| Mixture for | 5.11 | 3.01 | 4.68 |
| batch 2 | | | |

An appropriate amount of TPGDA is additionally added to batch 1.

### Formulation Examples

Batches 1-3 employed in the examples were characterized by HPLC-MS and MALDI-TOF. For mixtures 1 and 2 and also for the isolated OPUA 1 from batch 3, the weight fractions shown in Table 3 were obtained by GPC measurement.

TABLE 3
Composition and mass ratios of batches 1-3.

| | OPUA | UDA-IPDI | TPGDA | HEA |
|---|---|---|---|---|
| | [wt %] | [wt %] | [wt %] | [wt %] |
| Batch 1 (mixture 1) | 57 | 29 | 13 | 1 |
| Batch 2 (mixture 2) | 57 | 41 | — | 2 |
| Batch 3 (OPUA 1) | 100 | — | — | — |

### Isolation of an Oligomer Mixture OPUA 1

The oligomer mixture OPUA 1 is obtainable by filtration of mixture 1 through a column of silica gel. In a typical procedure, 33 g of mixture 1 in 12 ml of an eluent consisting of nheptane/ethyl acetate 60:40 (volume/volume) is loaded onto a column (diameter 4.8 cm, length 22 cm) of silica gel packed in eluent. Constituents other than OPUA 1 are eluted with 3.5 L of the same eluent. OPUA 1 is then washed from the column with 1.2 L of ethyl acetate. The product solution is carefully concentrated on a rotary evaporator with introduction of air. The free-flowing solution is applied in a thin layer and residual solvent is removed over a period of 17 days by allowing to stand in air at room temperature.

### Production of the Resins

For the production of resins, monomer mixtures were prepared according to the Tables 4-6 below and an initiator system added thereto (all figures are in wt %, in each case based on the total masses of the polymerizable dental material). The initiator system consisted in all examples of the same employed amounts of camphorquinone (CQ) and of 2-ethylhexyl p-dimethylaminobenzoate (EHA) as co-initiator. In all mixtures, 2,6-ditert-butyl-4-methylphenol (BHT) was used as stabilizer in the same concentration. The resulting resins were homogenized overnight with a stirrer.

Examples 1 to 8 in Tables 4 and 5 represent noninventive monomer mixtures and show the properties of monomer mixtures and polymerizable dental materials of the prior art. In comparative examples 3 to 8, the base monomer bis-GMA was replaced by other noninventive difunctional urethane (meth)acrylates. Although good volume shrinkage values are achieved in comparative examples 7 and 8, the flexural strength values are markedly poorer. Comparative examples 3, 4, 5 and 6 did not result in any improvement in either flexural strength or volume shrinkage.

TABLE 4
Composition, volume shrinkage, flexural strength, and elastic
modulus of noninventive monomer mixtures and corresponding
dental material compositions (comparative examples)

| | Comp. ex. 1 | Comp. ex. 2 | Comp. ex. 3 | Comp. ex. 4 |
|---|---|---|---|---|
| Bis-GMA [wt %] | 68.2 | 48.7 | | |
| UDMA [wt %] | | | 77.9 | 77.9 |
| X7261000 [wt %] | | | | |
| Exothane 8 [wt %] | | | | |
| Genomer 4277 [wt %] | | | | |
| MAU [wt %] | | | | 19.5 |
| TCDDMA [wt %] | | | | |
| TCDDA [wt %] | | | | |
| TEGDMA [wt %] | 29.2 | 48.7 | 19.5 | |
| CQ [wt %] | 1.0 | 1.0 | 1.0 | 1.0 |
| EHA [wt %] | 1.598 | 1.598 | 1.598 | 1.598 |
| BHT [wt %] | 0.002 | 0.002 | 0.002 | 0.002 |
| Total [wt %] | 100 | 100 | 100 | 100 |
| Base monomer M1 [wt %] | 0 | 0 | 0 | 0 |
| Base monomer M2 [wt %] | 0 | 0 | 0 | 0 |
| Base monomer M3 [wt %] | 0 | 0 | 77.9 | 97.4 |
| Other monomers OM [wt %] | 97.4 | 97.4 | 19.5 | 0 |
| Initiators, stabilizers [wt %] | 2.6 | 2.6 | 2.6 | 2.6 |
| Total [wt %] | 100 | 100 | 100 | 100 |
| Volume shrinkage [%] | 6.4 ± 0.3 | 8.2 ± 0.3 | 7.0 ± 0.6 | 6.9 ± 0.3 |
| Flexural strength [MPa] | 105 ± 8 | 96 ± 7 | 102 ± 4 | 94 ± 6 |
| Elastic modulus [GPa] | 2.6 ± 0.1 | 2.4 ± 0.2 | 2.1 ± 0.1 | 2.5 ± 0.1 |

TABLE 5
Composition, volume shrinkage, flexural strength, and elastic
modulus of noninventive monomer mixtures and corresponding
dental material compositions (comparative examples)

| | Comp. ex. 5 | Comp. ex. 6 | Comp. ex. 7 | Comp. ex. 8 |
|---|---|---|---|---|
| Bis-GMA [wt %] | | | | |
| UDMA [wt %] | 77.9 | | | |
| X7261000 [wt %] | | 77.9 | | |
| Exothane 8 [wt %] | | | 58.4 | |
| Genomer 4277 [wt %] | | | | 48.7 |
| MAU [wt %] | | | | |
| TCDDMA [wt %] | 19.5 | 19.5 | | |
| TCDDA [wt %] | | | 39.0 | 48.7 |
| TEGDMA [wt %] | | | | |
| CQ [wt %] | 1.0 | 1.0 | 1.0 | 1.0 |
| EHA [wt %] | 1.598 | 1.598 | 1.598 | 1.598 |
| BHT [wt %] | 0.002 | 0.002 | 0.002 | 0.002 |
| Total [wt %] | 100 | 100 | 100 | 100 |
| Base monomer M1 [wt %] | 0 | 0 | 0 | 0 |
| Base monomer M2 [wt %] | 0 | 19.5 | 39.0 | 48.7 |
| Base monomer M3 [wt %] | 97.4 | 77.9 | 58.4 | 48.7 |
| Other monomers OM [wt %] | 0 | 0 | 0 | 0 |
| Initiators, stabilizers [wt %] | 2.6 | 2.6 | 2.6 | 2.6 |
| Total [wt %] | 100 | 100 | 100 | 100 |
| Volume shrinkage [%] | 6.9 ± 0.3 | 6.7 ± 0.5 | 4.8 ± 0.2 | 4.1 ± 0.4 |
| Flexural strength [MPa] | 94 ± 6 | 25 ± 2 | 35 ± 1 | 52 ± 4 |
| Elastic modulus [GPa] | 2.5 ± 0.1 | 0.4 ± 0.1 | 0.7 ± 0.1 | 1.2 ± 0.1 |

Examples 9 to 14 in Table 6 correspond to monomer mixtures according to the invention and thus polymerizable dental materials according to the invention. In all examples, the volume shrinkage is markedly improved by comparison with the bis-GMA-TEGDMA resin mixtures (comparative examples 1 and 2) and the UDMA-TEGDMA resin mixture (comparative example 3). The measured flexural strength values for the inventive examples 9-14 are at least equivalent, in some cases even partially improved, by comparison with examples 1-3, even though shrinkage values are markedly reduced.

TABLE 6
Composition, volume shrinkage, flexural strength, and elastic modulus
of inventive monomer mixtures and dental material compositions

| | Ex. 9 | Ex. 10 | Ex. 11 | Ex. 12 | Ex. 13 | Ex. 14 |
|---|---|---|---|---|---|---|
| Mixture 1 | 77.9 | 62.3 | | | 39.0 | 29.2 |
| [wt %] | | | | | | |
| Mixture 2 | | | 48.7 | | | |
| [wt %] | | | | | | |
| OPUA 1 | | | | 48.7 | | |
| [wt %] | | | | | | |
| TCDDMA | 19.5 | 35.1 | 48.7 | | 19.4 | |
| [wt %] | | | | | | |
| TCDDA | | | | 48.7 | | 13.6 |
| [wt %] | | | | | | |
| UDMA | | | | | 39.0 | 54.6 |
| [wt %] | | | | | | |
| CQ | 1.0 | 1.0 | 1.0 | 1.0 | 1.0 | 1.0 |
| [wt %] | | | | | | |
| EHA | 1.598 | 1.598 | 1.598 | 1.598 | 1.598 | 1.598 |
| [wt %] | | | | | | |
| BHT | 0.002 | 0.002 | 0.002 | 0.002 | 0.002 | 0.002 |
| [wt %] | | | | | | |
| Total | 100 | 100 | 100 | 100 | 100 | 100 |
| [wt %] | | | | | | |
| Base | 44.4 | 35.5 | 27.7 | 48.7 | 22.2 | 16.6 |
| monomer M1 | | | | | | |
| [wt %] | | | | | | |
| Base | 19.5 | 35.1 | 48.7 | 48.7 | 19.4 | 13.6 |
| monomer M2 | | | | | | |
| [wt %] | | | | | | |
| Base | 22.6 | 18.1 | 20.0 | 0 | 50.3 | 63.1 |
| monomer M3 | UDA- | UDA- | UDA- | | UDA- | UDA- |
| [wt %] | IPDI | IPDI | IPDI | | IPDI/ | IPDI/ |
| | | | | | UDMA | UDMA |
| Other | 10.1 | 8.1 | 3.6 | 0 | 5.1 | 3.8 |
| monomers | TPGDA, | TPGDA, | 1 HEA | | TPGDA, | TPGDA, |
| OM [wt %] | 0.8 HEA | 0.6 HEA | | | 0.4 HEA | 0.3 HEA |
| Initiators, | 2.6 | 2.6 | 2.6 | 2.6 | 2.6 | 2.6 |
| stabilizers | | | | | | |
| [wt %] | | | | | | |
| Total | 100 | 100 | 100 | 100 | 100 | 100 |
| [wt %] | | | | | | |
| Volume | 4.7 ± 0.2 | 4.7 ± 0.3 | 5.3 ± 0.1 | 5.1 ± 0.1 | 5.3 ± 0.3 | 5.3 ± 0.1 |
| shrinkage | | | | | | |
| [%] | | | | | | |
| Flexural | 105 ± 4 | 110 ± 12 | 105 ± 11 | 90 ± 3 | 111 ± 1 | 114 ± 6 |
| strength | | | | | | |
| [MPa] | | | | | | |
| Elastic | 2.3 ± 0.1 | 2.6 ± 0.3 | 2.9 ± 0.1 | 1.9 ± 0.2 | 2.7 ± 0.1 | 2.5 ± 0.2 |
| modulus | | | | | | |
| [GPa] | | | | | | |

### Production of Dental Composites

Dental composites were produced according to Table 7 below. In examples 15-19, inventive monomer mixtures were used. In comparative example 20, a corresponding dental composite was produced with a monomer mixture comprising bis-GMA. For the production of the dental composites, a total of 75% by weight of dental glass G018-053 from Schott AG (median grain size 0.7 μm, 6% by weight of silane) based on the total mass of the dental composite was added in stages to the resin mixtures (dental compositions) obtained above, with homogenization using a SpeedMixer DAC 400-1 VAC-P (Hauschild, Germany). The mixture was then degassed at 20 mbar for 3 min with further mixing.

TABLE 7
Composition, volume shrinkage, flexural strength, and elastic modulus of inventive
dental composites having a filler content of 75% by weight (examples 15-19)

| | Ex. 15 | Ex. 16 | Ex. 17 | Ex. 18 | Ex. 19 |
|---|---|---|---|---|---|
| Mixture 1 [wt %] | | | 62.3 | 48.7 | |
| Mixture 2 [wt %] | | 48.7 | | | 58.4 |
| OPUA [wt %] | 48.7 | | | | |
| TCDDMA [wt %] | 48.7 | 48.7 | 35.1 | 48.7 | |
| TCDDA [wt %] | | | | | |
| TCDA [wt %] | | | | | 39.0 |
| Bis-GMA [wt %] | | | | | |
| TEGDMA [wt %] | | | | | |
| CQ [wt %] | 1.0 | 1.0 | 1.0 | 1.0 | 1.0 |
| EHA [wt %] | 1.598 | 1.598 | 1.598 | 1.598 | 1.598 |
| BHT [wt %] | 0.002 | 0.002 | 0.002 | 0.002 | 0.002 |
| Total [wt %] | 100 | 100 | 100 | 100 | 100 |
| Base monomer M1 [wt %] | 48.7 | 27.8 | 35.5 | 27.8 | 33.3 |
| Base monomer M2 [wt %] | 48.7 | 48.7 | 35.1 | 48.7 | 39.0 |
| Base monomer M3 [wt %] | | 20.0 | 18.1 | 14.1 | 23.9 |
| | | UDA-IPDI | UDA-IPDI | UDA-IPDI | UDA-IPDI |
| Other monomers OM [wt %] | | 1.0 HEA | 8.1 TP-GDA | 6.3 TP-GDA | 1.2 HEA |
| | | | 0.6 HEA | 0.5 HEA | |
| Initiators, stabilizers [wt %] | 2.6 | 2.6 | 2.6 | 2.6 | 2.6 |
| Total [wt %] | 100 | 100 | 100 | 100 | 100 |
| Volume shrinkage [%] | 2.0 ± 0.1 | 2.5 ± 0.1 | 2.6 ± 0.1 | 2.4 ± 0.1 | 2.0 ± 0.3 |
| Flexural strength [MPa] | 129 ± 3 | 147 ± 6 | 126 ± 11 | 126 ± 13 | 151 ± 7 |
| Elastic modulus [GPa] | 10.5 ± 1.5 | 10.7 ± 0.3 | 9.7 ± 0.6 | 10.3 ± 0.6 | 10.0 ± 1.0 |

TABLE 8
Composition, volume shrinkage, flexural strength, and elastic
modulus of a noninventive dental composite having a filler
content of 75% by weight (comparative example 20)

| | Comp. ex. 20 |
|---|---|
| Mixture 1 [wt %] | |
| Mixture 2 [wt %] | |
| OPUA [wt %] | |
| TCDDMA [wt %] | |
| TCDDA [wt %] | |
| TCDA [wt %] | |
| Bis-GMA [wt %] | 48.7 |
| TEGDMA [wt &] | 48.7 |
| CQ [wt %] | 1.0 |
| EHA [wt %] | 1.598 |
| BHT [wt %] | 0.002 |
| Total [wt %] | 100 |
| Base monomer M1 [wt %] | 0 |
| Base monomer M2 [wt %] | 0 |
| Base monomer M3 [wt %] | |
| Other monomers OM [wt %] | 97.4 bis-GMA/TEGDMA |
| Initiators, stabilizers [wt %] | 2.6 |
| Total [wt %] | 100 |
| Volume shrinkage [%] | 3.3 ± 0.2 |
| Flexural strength [MPa] | 132 ± 11 |
| Elastic modulus [GPa] | 10.1 ± 0.3 |

### Determination of Flexural Strength and Elastic Modulus

Flexural strength and elastic modulus were determined. The test specimens for this were produced in accordance with ISO 4049:2009. In a departure therefrom, the test specimens were produced by irradiating with a HiLite® power light-polymerization device (Heraeus). For this, the dental composites in the test specimen shapes (40 mm×2 mm×2 mm) were each irradiated from both sides for 90 s. The test specimens were stored for 24 hours in distilled water at 37° C. The flexural strength and elastic modulus were determined using a Zwick universal testing machine (model Z010 or Z2.5, Zwick-Roell, Germany). The average value of 6 individual measurements and the standard deviation are reported.

### Volume Shrinkage

The volume shrinkage was calculated from the difference in density ρ of the dental composites before (VA) and 24 hours after (NA) curing. 3 samples were measured per composite and the average value was used as the density. For the determination of the density of the composites after curing, cylindrical test specimens (8 mm in diameter and 2 mm in height) were produced by illuminating with a HiLite power light-polymerization device (Heraeus). The illumination was carried out for 90 s from both sides of the test specimen. These were stored dry for 24 hours at 23° C. The density of the cured and uncured composites was measured using a helium gas pycnometer (AccuPyc III 1340, Micromeritics Instrument Corporation, USA, GA).

The volume shrinkage VS was determined from the following formula:

$$VS = 100 \% \times ( {ρ}_{NA} - {ρ}_{V A} ) / {ρ}_{NA} .$$

### GPC Measurement

The measurement was carried out on a GPC system (PSS SECcurity GPC system, from PSS Polymer Standards Service GmbH, Germany) with column oven and RI detector.

The separation of the constituents employed the following column combination: guard column VA 50/7.7 Nucleogel GP 5 P, separation columns VA 300/7.7 Nucleogel GPC 104-5, and VA 300/7.7 Nucleogel GPC 500-5 (all from Macherey & Nagel). The columns were thermostatically controlled at 20° C.

The sample concentration was approx. 1%. 20 μl of each sample was injected and THF ((Merck 109731) was used as mobile phase. The flow rate of the mobile phase was 0.5 ml/min.

The proportions of oligomers and monomers were determined by integrating the respective areas under the measurement curve of the refractive index detector; in the case of overlapping peaks, the perpendicular was dropped onto the volume axis at the local minimum of the curve between the peaks and the intersection of the perpendicular with the volume axis used as the integration limit.

1. A monomer mixture for producing a dental material, comprising:
a. at least one base monomer M1 of the following empirical formula 1: 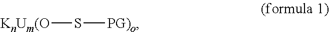
where
PG=is a polymerizable group selected from —OOC—C═CH₂ and —OOC—C(CH₃)═CH₂;
S=is a spacer group selected from unbranched and branched alkylene having 1-10 carbon atoms, which may additionally contain oxygen and/or —OOC— in the carbon chain, and is preferably ethylene;
O=is oxygen;
U=is a group represented by the following formula 2: 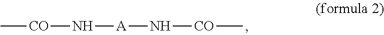
where
A=is a group selected from a divalent aromatic or aliphatic C6-C20 hydrocarbon group, preferably a divalent C6-C13 aliphatic hydrocarbon group, more preferably a divalent saturated, cyclic C6-C13 hydrocarbon group;
K=is a group represented by the following formula 3: 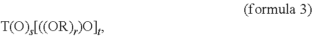
where
T=is a trivalent hydrocarbon group having 3-7 carbon atoms,
O=is oxygen,
R=is in each case independently selected from an ethylene group, a 1,2-propylene group, a 1,3-propylene group and a mixture thereof, preferably a 1,2-propylene group,
r=is in each case independently 1-12, preferably 1-9, more preferably 1-6,
s=is 0 or 1, preferably 0,
t=is 2 or 3, preferably 3,
where the condition must be met that s+t=3;
n=1-9, preferably 1-7, more preferably 1-5;
where the condition must be met that m=2n+1 and o=n+2;
b. at least one base monomer M2 of the following formula 4: 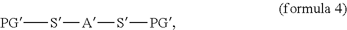
where
PG′=is a polymerizable group selected from —OOC—CH═CH₂ and —OOC—C(CH₃)═CH₂;
S′=is a spacer group selected from unbranched and branched alkylene having 1-10 carbon atoms, which may additionally contain oxygen and/or —OOC— in the carbon chain, and is preferably methylene;
or S′ is absent;
A′=is an aliphatic polycyclic group, preferably an aliphatic tricyclic hydrocarbon group, in which one or more hydrogen atoms may each be independently replaced by C1-C4 alkyl radicals, C1-C4 alkoxy radicals, fluorine atoms, chlorine atoms or trifluoromethyl groups, more preferably is tricyclodecanylene, even more preferably tricyclo[5.2.1.0/2,6]decanylene.

2. The monomer mixture as claimed in claim 1, characterized in that T is a trivalent hydrocarbon group having 3 carbon atoms and is preferably represented by the following formula 5: 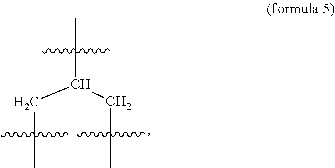 [SMILES: *CC(*)C*]
where the indicated bonds each represent the points of attachment to the oxygen atoms in formula 3.

3. The monomer mixture as claimed in claim 1 or 2, characterized in that A is a divalent hydrocarbon group having 10 carbon atoms and is preferably represented by the following formula 6: 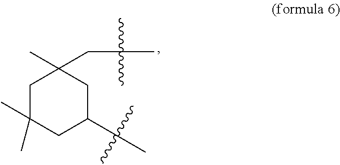 [SMILES: *CC1(C)CC(*)CC(C)(C)C1]
where the two indicated bonds each represent the points of attachment to the nitrogen atoms in formula 2.

4. The monomer mixture as claimed in any of claims 1 to 3, characterized in that, in the monomer mixture, a plurality of base monomers M1, preferably at least two base monomers M1, more preferably more than two base monomers M1, even more preferably more than three base monomers M1, even more preferably more than four base monomers M1, are present.

5. The monomer mixture as claimed in any of claims 1 to 4, characterized in that the base monomer M2 is selected from bis(methacryloyloxymethyl)tricyclo[5.2.1.0/2,6]decane, bis(acryloyloxymethyl)tricyclo[5.2.1.0/2,6]decane, and mixtures thereof and that preferably the base monomer M2 is bis(acryloyloxymethyl)tricyclo[5.2.1.0/2,6]decane.

6. The monomer mixture as claimed in any of claims 1 to 5, characterized in that the monomer mixture comprises a base monomer M3 that differs from the base monomers M1 of the formula 1 and M2 of the formula 2, and the base monomer M3 is preferably selected from urethane-based monomers, even more preferably the base monomer M3 is selected from UDMA, UDA, UDA-IPDI, and mixtures thereof.

7. The monomer mixture as claimed in any of claims 1 to 6, characterized in that one or more of the following base monomers are present in the following mass fractions based on the total mass of the monomer mixture:
base monomer M1 from 2% to 75% by weight, preferably from 5% to 68% by weight, more preferably from 13% to 63% by weight, even more preferably from 15% to 52% by weight;
base monomer M2 from 5% to 96% by weight, preferably from 12% to 65% by weight, more preferably from 30% to 63.5% by weight, even more preferably from 30% to 52% by weight;
base monomer M3 from 0% to 75% by weight, preferably from 0.1% to 65% by weight, more preferably from 12% to 65% by weight, even more preferably from 12% to 64% by weight.

8. The monomer mixture as claimed in any of claims 1 to 7, characterized in that the monomer mixture does not contain any monomer having a bisphenol A structure and that preferably 2,2-bis[4-(2-hydroxy-3-(meth)acryloyloxypropoxy)phenyl]propane (bis-GMA) and ethoxylated bisphenol A di(meth)acrylate (bis-EMA) are absent.

9. The monomer mixture as claimed in any of claims 1 to 8, characterized in that the monomer mixture does not contain any monomer selected from low-molecular-weight and low-viscosity mono- and di(meth)acrylates, any monomer having a viscosity at a temperature of 23° C. of less than 0.05 Pa·s and/or having partial solubility in water and/or any monomer selected from hexanediol diacrylate (HDDA), hexanediol dimethacrylate (HDDMA), triethylene glycol diacrylate (TEGDA), and triethylene glycol dimethacrylate (TEGDMA).

10. A monomer mixture for producing a dental material, comprising:
a. at least two or more base monomers M1 represented by the following empirical formula 1: 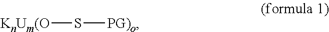
where
PG=is a polymerizable group selected from —OOC—CH═CH₂ and —OOC—C(CH₃)═CH₂;
S=is a spacer group selected from unbranched and branched alkylene having 1-10 carbon atoms, which may additionally contain oxygen and/or —OOC— in the carbon chain, and is preferably ethylene;
O=is oxygen;
U=is a group represented by the following formula 2: 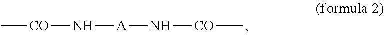
where
A=is a group selected from a divalent aromatic or aliphatic C6-C20 hydrocarbon group, preferably a divalent C6-C13 aliphatic hydrocarbon group, represented by the following formula 6: 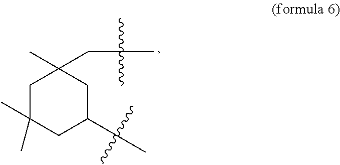 [SMILES: *CC1(C)CC(*)CC(C)(C)C1]
where the two indicated bonds each represent the points of attachment to the nitrogen atoms in formula 2;
K=is a group represented by the following formula 3: 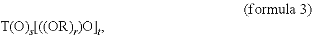
where
T=is a trivalent hydrocarbon group having 3-7 carbon atoms, preferably a trivalent hydrocarbon group, represented by the following formula 5: 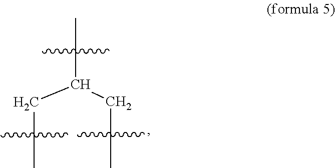 [SMILES: *CC(*)C*]
where the three indicated bonds each represent the points of attachment to the oxygen atoms in formula 3,
O=is oxygen,
R=is in each case independently selected from an ethylene group, a 1,2-propylene group, a 1,3-propylene group and a mixture thereof, preferably a 1,2-propylene group,
r=is in each case independently 1-12, preferably 1-9, more preferably 1-6,
s=is 0 or 1, preferably 0,
t=is 2 or 3, preferably 3,
where the condition must be met that s+t=3;
n=1-9, preferably 1-7, more preferably 1-5;
where the condition must be met that m=2n+1 and o=n+2;
b. optionally at least one base monomer M2 of the following formula 4: 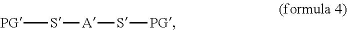
where
PG′=is a polymerizable group selected from —OOC—CH═CH₂ and —OOC—C(CH₃)═CH₂;
S′=is a spacer group selected from unbranched and branched alkylene having 1-10 carbon atoms, which may additionally contain oxygen and/or —OOC— in the carbon chain, and is preferably methylene;
or S′ is absent;
A′=is an aliphatic polycyclic group, preferably an aliphatic tricyclic hydrocarbon group, in which one or more hydrogen atoms may each be independently replaced by C1-C4 alkyl radicals, C1-C4 alkoxy radicals, fluorine atoms, chlorine atoms or trifluoromethyl groups, more preferably is tricyclodecanylene, even more preferably tricyclo[5.2.1.0^{2,6}]decanylene.

11. The use of the monomer mixture according to any one of claims 1 to 10 for the production of a polymerizable dental material, preferably a dental composite, core build-up material, root-canal filling material, filling material, underfill material, fixation material, crown material, bridge material, restoration material and/or prosthesis material.

12. A polymerizable dental material, comprising:
a) a monomer mixture as claimed in any of claims 1 to 10;
b) optionally at least one initiator or initiator system for the polymerization;
c) optionally fillers;
d) optionally customary dental additives.

13. The dental material as claimed in claim 12, characterized in that one or more of the following constituents are present in the dental material in the following mass fractions based on the total mass of the dental material:
a) the monomer mixture from 5% to 99% by weight, preferably from 10% to 95% by weight, more preferably from 15% to 85% by weight;
b) the at least one initiator or initiator system for the polymerization from 0% to 5% by weight, preferably from 0.01% to 5% by weight;
c) the fillers from 0% to 95% by weight, preferably from 1% to 95% by weight, more preferably from 5% to 90% by weight, even more preferably from 15% to 85% by weight;
d) the customary dental additives from 0% to 5% by weight, preferably from 0.001% to 5% by weight.

14. The dental material as claimed in either of claim 12 or 13 for use in a treatment process as a dental composite, filling material, underfill material, fixation material, core build-up material, root-canal filling material, crown material, bridge material, restoration material and/or prosthesis material.

15. A cured dental material produced from a polymerizable dental material as claimed in either of claim 12 or 13.
